(19) United States
(12) Patent Application Publication
(10) Pub. No.: US 2020/0008840 A1
(43) Pub. Date: Jan. 9, 2020

(54) UTERINE LAVAGE DEVICES, SYSTEMS, AND METHODS

(71) Applicant: Previvo Genetics, Inc., San Carlos, CA (US)

(72) Inventors: Bruce ADDIS, San Carlos, CA (US); Sam NAJMABADI, Beverly Hills, CA (US); Steven NAKAJIMA, Broomfield, CO (US)

(21) Appl. No.: 16/505,304

(22) Filed: Jul. 8, 2019

Related U.S. Application Data
(60) Provisional application No. 62/695,626, filed on Jul. 9, 2018.

(51) Int. Cl. A61B 17/435; A61M 3/02; A61B 1/05; A61B 1/303; A61B 1/06; A61M 25/00; A61M 11/00

(52) U.S. Cl. CPC A61B 17/435; A61M 3/0283; A61B 1/05; A61M 2025/0039; A61B 1/0676; A61M 25/007; A61M 11/00; A61B 1/303

(57) ABSTRACT

Uterine lavage devices and methods are described. A uterine lavage catheter device for recovering oocytes or blastocysts from a human uterus may include a seal element configured to provide a sealing surface against the exterior cervical ostium, a supply lumen extending from the seal element and configured to supply lavage fluid, a nozzle configured to generate a spray pattern, and a return lumen.

### CROSS-REFERENCE TO RELATED APPLICATIONS AND INCORPORATION BY REFERENCE

This application claims priority to U.S. Provisional Patent Application No. 62/695,626, filed on Jul. 9, 2018, which is incorporated by reference herein in its entirety for all purposes.

### FIELD

The described embodiments relate generally to uterine lavage devices, systems and methods. More particularly, the present embodiments relate to uterine lavage catheter devices, systems, and methods.

### BACKGROUND

Several parameters are important when considering uterine lavage catheter systems. For example, ensuring that the device is minimally invasive is of high importance. Additionally, it is important to provide efficient recovery of in vivo developed embryos from the uterus of a patient, prior to implantation in the uterine wall. In this way, the embryos, once recovered, can be screened for various conditions (such as specific genetic diseases). Moreover, the recovered embryos may be cryopreserved and replaced at a later time. Ensuring the gentle recovery of naturally fertilized and incubated embryos, prior to implantation in the uterine wall, presents challenges that have been addressed herein.

### SUMMARY

In general, at a time when a woman's uterus contains in vivo fertilized preimplantation embryos, a seal is provided, between the uterus and the external environment, against flow of fluid from the uterus to the external environment. While the seal is provided, fluid is delivered past the seal and into the uterus. The delivered fluid is withdrawn, with the embryos, past the seal and from the uterus to the external environment.

The recovered in vivo pre-implantation embryos are recovered for genetic diagnosis or genetic therapy or sex determination or any combination thereof. One or more of the embryos are returned to the uterus of the woman. The one or more embryos are returned to the uterus of the woman with or without having frozen the embryos. The embryos result from natural or from artificial insemination. Superovulation may be applied in this process. One or more preimplantation embryos may be treated, e.g., with gene therapy.

The delivering or withdrawing or both of the fluid is pulsatile. The fluid is withdrawn while the seal is being provided. The seal enables essentially all of the fluid to be withdrawn. The withdrawing of fluid includes aspirating the fluid from the uterus. Both the delivering and the withdrawing are pulsatile and, in one embodiment, the pulses of the delivered fluid and the withdrawn fluid are coordinated.

Some embodiments are directed to a uterine lavage catheter device for recovering blastocysts from a human uterus. The catheter device includes a seal element configured to provide a sealing surface against the exterior cervical ostium, a supply lumen extending from the seal element and configured to supply lavage fluid, a nozzle configured to generate an asymmetric spray pattern, the nozzle coupled to the supply lumen, and a return lumen having an inlet, the return lumen disposed coaxially with the supply lumen. The inlet of the return lumen is located a first distance from the sealing surface, such that the inlet to the return lumen is configured to be placed at the interior cervical ostium. In some embodiments, the seal element is a conical distal surface that provides sealing.

In some embodiments, the catheter device includes an extension element extending from and in contact with the seal element. The extension element further includes a distal surface that includes the sealing surface, the extension element configured to shorten the first distance. The distal surface of the extension element is conical in shape.

In some embodiments, the catheter device defines a second distance between the distal end of the nozzle and the inlet to the return lumen, and the supply lumen is translatable along the shared lumen axis between about 1 cm and 7 cm. In some embodiments, the catheter device defines a second distance between the distal end of the nozzle and the inlet to the return lumen, and the supply lumen is translatable along the shared lumen axis between about 3 cm and 5 cm. In some embodiments, the catheter device defines a second distance between the distal end of the nozzle and the inlet to the return lumen, and the supply lumen is translatable along the shared lumen axis between about 1 mm and 5 mm. The supply lumen is rotatable along the shared lumen axis. In some embodiments, the catheter device defines a second distance between the sealing surface and the tip of the nozzle; and a third distance between the sealing surface and the inlet of the suction line such that the difference between the second and third distances is adjustable between about 1 cm and 7 cm, or between about 3 cm and 5 cm. In some embodiments, the return lumen is the sole suction path for fluid supplied by the fluid supply lumen and blastocysts. The return lumen is steerable between about −60 degrees and +60 degrees off-axis. In some embodiments, the return lumen is the sole suction path for fluid supplied by the fluid supply lumen and blastocysts. The return lumen is steerable between about −40 degrees and +40 degrees off-axis. In some embodiments, the return lumen is the sole suction path for fluid supplied by the fluid supply lumen and blastocysts. The return lumen is steerable between about −10 degrees and +10 degrees off-axis.

In some embodiments, the nozzle further includes a first fluid outlet and a second fluid outlet angularly offset from the first fluid outlet with respect to the axis of the fluid supply lumen. The angle between the first fluid outlet and the second fluid outlet is between about 0 degrees and 60 degrees. The nozzle may include a first fluid outlet disposed between about 5 degrees and 25 degrees off-axis. The nozzle may include a second fluid outlet disposed between about 25 and 55 degrees off-axis. The fluid outlets may be angled from one another with respect to the axis of the fluid supply lumen. The fluid outlets may also have different sizes or shapes.

In some embodiments, the catheter device includes a housing enclosing a portion of the fluid supply lumen and the return lumen. The fluid supply lumen exits the return lumen along a first housing axis, and the return lumen exits the housing in the annular space. In some embodiments, the return lumen includes a radius of curvature between the first housing axis and second housing axis such that recovered blastocysts freely flow through the return lumen. In some embodiments, the fluid supply lumen exits the return lumen in a portion of the radius of curvature. The radius of curvature is between about 25 mm and 100 mm.

In some embodiments, the nozzle includes a proximal sealing surface configured to seal the inlet of the return lumen when the supply lumen is in a first position. The proximal sealing surface extends into the inlet of the return lumen when the supply lumen is in the first position, in some embodiments. In some embodiments, the proximal sealing surface is conical in shape.

Some embodiments are directed to a method of recovering blastocysts from a human uterus. The method may include inserting a catheter of the uterine lavage device trans-vaginally into a uterus, sealing the exterior cervical ostium with a sealing surface of a seal element, lavaging the uterine walls with lavage fluid with a nozzle, the nozzle coupled to a supply lumen of the catheter device. The method may include placing an inlet of a return lumen at the interior cervical ostium by setting a distance between the sealing surface and the inlet of the return lumen, the return lumen positioned coaxially with the supply lumen, and recovering the lavage fluid and blastocysts from the uterus with a return lumen disposed coaxially with a supply lumen that supplies the fluid to the nozzle. In some embodiments, the method may include translating the nozzle coaxially along the longitudinal axis of the catheter device between about 1 cm and 5 cm. In some embodiments, the method may include rotating the nozzle about the longitudinal axis of the catheter device such that a first and second fluid outlet are sprayed in a concentric circular pattern. In some embodiments, the method may include bending the return lumen off-axis from the longitudinal axis of the catheter device between about +60 degrees and −60 degrees. In some embodiments, the method may include flowing the lavage fluid through the return lumen through a radius of curvature that intersects a point through which the supply lumen exits the return lumen. In some embodiments, the method may include illuminating a portion of the uterus with a light source coupled to the nozzle, and imaging, via a camera coupled to the nozzle, a portion of the uterus that is illuminated.

Some embodiments are directed to a method of recovering blastocysts from a human uterus. The method may include inserting a catheter of the uterine lavage device trans-vaginally into a uterus, sealing the exterior cervical ostium with a sealing surface of a seal element, and lavaging the uterine walls with lavage fluid with a nozzle at a first fluid pressure, wherein the lavaging comprises pulsing lavage fluid between the first fluid pressure and a second fluid pressure different than the first fluid pressure. The method may include placing an inlet of a return lumen at the interior cervical ostium, and recovering the lavage fluid and blastocysts from the uterus with the return lumen, wherein the first fluid pressure is between about 25 mmHg and 75 mmHg. In other embodiments, the first fluid pressure may range between 20 PSI and 80 PSI. In some embodiments, the first fluid pressure may be about 40 PSI. In some embodiments, the first fluid pressure may be about 50 PSI.

Some embodiments are directed to a uterine lavage catheter device for recovering blastocysts from a human uterus, including a seal element configured to provide a sealing surface against the exterior cervical ostium, a supply lumen extending from the seal element and configured to supply lavage fluid, a nozzle in fluid communication with the supply lumen and configured to generate a spray pattern, a return lumen, and a tip direction fitting, configured to be coupled to one of the supply lumen or return lumen, the tip direction fitting providing a pre-bend to the catheter such that the angle of the catheter tip is fixed and offset (e.g., angled) from the longitudinal axis of the catheter.

Some embodiments are directed to a uterine lavage catheter device for recovering blastocysts from a human uterus, including a seal element configured to provide a sealing surface against the exterior cervical ostium, a supply lumen extending from the seal element and configured to supply lavage fluid, a nozzle in fluid communication with the supply lumen and configured to generate a spray pattern, a return lumen, and an imaging sensor configured to image a portion of the uterus. In some embodiments, the imaging sensor comprises a CCD sensor. The imaging sensor is integrated into the nozzle in some embodiments. In some embodiments, the imaging sensor is integrated into a distal end of return lumen. The catheter device may further include a light source, the light source configured to illuminate a portion of the uterus configured to be imaged by the imaging sensor.

Some embodiments are directed to an uterine lavage catheter device for recovering blastocysts from a human uterus, including a seal element including a sealing surface configured to be disposed against the external cervical ostium; a supply lumen extending from the seal element and configured to supply lavage fluid; a nozzle coupled to the supply lumen; a return lumen having an inlet, the return lumen disposed coaxially with the supply lumen; and an extension assembly operatively connected to the seal element and configured to move the seal element in a longitudinal direction along the uterine lavage catheter device to shorten a distance between the inlet of the return lumen and an interior cervical ostium of the human uterus.

Some embodiments are directed to an uterine lavage catheter device for recovering blastocysts from a human uterus, including a seal element including a sealing surface configured to be disposed against a cervix surface surrounding an exterior cervical ostium of the human uterus; a supply lumen extending from the seal element and configured to supply lavage fluid; a nozzle coupled to the supply lumen; a return lumen having an inlet, the return lumen disposed coaxially with the supply lumen; and a vacuum lumen connected to the vacuum port and in communication with a vacuum source, wherein the vacuum lumen is configured to exhaust air from the seal element such that the sealing surface is configured to be pressure fitted against the cervix surface surrounding the exterior cervical ostium of the human uterus.

Some embodiments are directed to an uterine lavage catheter system for recovering blastocysts from a human uterus, including: a uterine lavage catheter device, a collection container, and an elevator. The catheter device comprises a seal element comprising a sealing surface configured to be disposed against a cervix surface surrounding an exterior cervical ostium of the human uterus; a supply lumen extending from the seal element and configured to supply lavage fluid; a nozzle coupled to the supply lumen; a return lumen having an inlet, the return lumen disposed coaxially with the supply lumen. The collection container is disposed external to the catheter device and defines a fluid head to control intrauterine pressure and uterine expansion of the human uterus. The elevator comprises a track and a cradle coupled to the track and configured to receive and hold the container, wherein the elevator is configured to raise or lower the cradle holding the collection container along the track to adjust the intrauterine pressure during the lavage procedure.

In some embodiments, the lavage system may be used to recover oocytes from the uterus for fertility preservation. In various embodiments, the lavage procedure described herein may be performed within 0 to 36 hours of natural ovulation (or within 0 to 24 hours, 12 to 36 hours, or 24 to 36 hours of natural ovulation) to recover oocytes that are present in the uterus. In various embodiments, the lavage procedure described herein may be performed within 36 to 72 hours after the application of an ovulatory trigger (or within 36 to 60 hours, 48 to 72 hours, or 48 to 60 hours after application of an ovulatory trigger) to recover oocytes that are present in the uterus. Superovulation may be applied in this process similar to superovulation process of in vivo embryo recovery, however without an artificial intrauterine insemination.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows a schematic view of a female reproductive tract undergoing superovulation.

FIG. 2 shows a schematic view of a female reproductive tract undergoing artificial insemination.

FIG. 3 shows a schematic view of a female reproductive tract undergoing uterine lavage, and shows a portion of a uterine lavage catheter device according to an embodiment.

FIG. 4 shows a simplified sectional view of a uterine lavage catheter device according to an embodiment.

FIG. 5 shows an enlarged distal end of a uterine lavage catheter device according to an embodiment.

FIG. 6 shows schematic illustrations of a catheter device steering system according to an embodiment.

FIG. 7 shows an schematic sectional view of a manifold design according to an embodiment.

FIG. 8 shows an enlarged view of the manifold design shown in FIG. 7.

FIGS. 9A and 9B show a comparative illustrations of manifold designs.

FIG. 10 shows a schematic view of a female reproductive tract.

FIG. 11 shows an enlarged view of a distal end of a uterine lavage catheter device utilizing an extension element according to an embodiment.

FIG. 12 shows a tip direction fitting according to an embodiment.

FIG. 13 shows an imaging system according to an embodiment.

FIG. 14 shows a schematic view of stagnating flow with parked cells.

FIG. 15 shows a schematic side view of an uterine lavage catheter device according to an embodiment.

FIG. 16 shows an enlarged schematic view of an actuator for extension assembly of the uterine lavage catheter device according to an embodiment.

FIG. 17 shows a schematic cross-sectional view taken along line A-A of FIG. 16 according to an embodiment.

FIG. 18 shows a detailed schematic view of an actuator for extension assembly of the uterine lavage catheter device according to an embodiment.

FIG. 19 shows a schematic side view of an uterine lavage catheter device according to an embodiment.

FIG. 20 shows an enlarged schematic view of a seal element of the uterine lavage catheter device according to an embodiment.

FIG. 21 an enlarged schematic view of a seal element of the uterine lavage catheter device according to an embodiment.

FIG. 22 shows a cross-sectional schematic view of the uterine lavage catheter device according to an embodiment.

FIG. 23 shows a side schematic view of a ratchet tenaculum stabilizer according to an embodiment.

FIG. 24 shows a perspective schematic view of a ratchet tenaculum stabilizer according to an embodiment.

FIG. 25 shows a detailed side view of a ratchet tenaculum stabilizer according to an embodiment.

FIG. 26 shows a perspective schematic view of a ratchet tenaculum stabilizer with a spring clutch assembly according to an embodiment.

FIG. 27 shows a cross-sectional schematic view of a spring clutch assembly according to an embodiment

FIG. 28 shows an imagery system disposed at an inlet of return lumen of uterine lavage catheter device according to an embodiment.

FIG. 29 shows an elevator for lifting and lowering a collection bottle for a collection system according to an embodiment.

FIG. 30A shows a toothed-belt assembly of an elevator according to an embodiment.

FIG. 30B shows a screw-drive assembly of an elevator according to an embodiment.

FIG. 30C shows a rack-gear assembly of an elevator according to an embodiment.

FIG. 31 shows a uterine lavage catheter device with a tenaculum stabilizer according to an embodiment.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

As discussed above, medical catheter device design has evolved over the years in response to new surgical and clinical techniques, new novel materials, better design for manufacturing, better design for use, more informed safety considerations, and the like. Additionally, when new and paradigm shifting medical procedures are conceived, opportunities for novel catheter device design systems, methods, and the like, are ripe. Human uterine lavage is such a medical procedure. In general, uterine lavage offers a new, natural-like choice in assisted fertility reproduction.

Several parameters are important when considering uterine lavage catheter device systems. For example, ensuring that the device is minimally invasive is of high importance. Additionally, it is important to provide efficient recovery of in vivo developed embryos from the uterus of a patient, prior to implantation in the uterine wall. In this way, the embryos, once recovered, can be screened for various conditions (such as specific genetic diseases). Moreover, the recovered embryos may be cryopreserved and replaced at a later time. Ensuring the gentle recovery of naturally fertilized and incubated embryos, prior to implantation in the uterine wall, presents challenges that have been addressed herein.

By ensuring comfortable, relatively noninvasive harvesting of embryos, preimplantation diagnostics may be a more attractive option for women and couples considering having children. These systems and methods provide a simple, safe, and inexpensive way to diagnose and treat human embryos before implantation (e.g., preimplantation genetic diagnosis, “PGD”, testing for aneuploidy “PGT-A”), preimplantation genetic screening, (“PGS,”” preimplantation genetic testing for monogenic/single gene disorder, “PGT-M”) and preimplantation genetic testing for chromosome structural arrangements, “PGT-SR”) for determination of possible aneuploidy status, or to make a sex determination, for example. Compared to in vitro fertilization (IVF), PGS and/or PGD by uterine lavage is expected to be less expensive, less technically difficult, and more cost efficient than PGS and/or PGD using IVF.

In the same vein, by ensuring systems and methods that are user-friendly for physicians, wide-ranging adoption of such technologies becomes more likely. Leveraging human factors engineering, in combination with a new process, allows for more efficient embryo retrieval, and in turn may allow for higher success rates overall.

The uterine lavage catheter devices and systems described in this document achieve these and other beneficial characteristics by balancing application of lavage fluid in particular patterns and through motion of the supply lumen, optimizing the collection of lavage fluid and cells, improving flow of lavage fluid both in the supply lumen and collection lumen, and providing additional accessories for use with the systems and methods. In order to retrieve embryos at various stages such as blastocysts, oocytes, or microspheres, a supply lumen provides lavage fluid to a nozzle that sprays the fluid onto the uterine wall. Certain structural flexibility including translation rotation and bending directionality allow a physician greater choice as to an optimum lavage pattern. Additionally, the use of asymmetric spray patterns such as plural fluid outlets disposed offset from one another on the nozzle allow for a larger display area to be covered for a given nozzle surface area. Structural features described herein allow for controlling where the inlet of the return lumen is located in a given patient while providing a sealing surface to seal the uterus such that collection is precise. The return lumen includes fluidic features that allow for smooth collection of lavage fluid and embryos. Finally, accessory elements and modules are described that provide advantages to the uterine lavage catheter device systems and methods described herein, such as tenaculum stabilizers, extension elements, static directional catheter tips, catheter device imaging systems, and fluid head defined by a collection container for controlling intrauterine pressure and uterine expansion.

These features balance each of the aforementioned goals—including patient safety and comfort, physician comfort and usability, and embryo collection success and efficiency.

The catheter device systems described herein may be referred to as uterine lavage catheter devices. However, other applications of these catheter devices and methods described are envisioned, for example in other applications where fluid lavage and lavage fluid collection are desired.

These and other embodiments are discussed below with reference to the accompanying figures. However, those skilled in the art will readily appreciate that the detailed description given herein with respect to these figures is for explanatory purposes only and should not be construed as limiting.

In some embodiments, very early diagnosis and treatment of genetic disorders in human preimplantation embryos (blastocysts) conceived in vivo and recovered from the reproductive tracts of fertile women are contemplated. Important beneficiaries of what we describe here are women who, are single or in specific unions with their male or female partners, are faced with parenting yet-to-be-born children at (significant) risk for childhood or adult-onset genetic diseases, or those women who are seeking fertility preservation, or have experienced a history of recurrent pregnancy loss, and/or advanced maternal age.

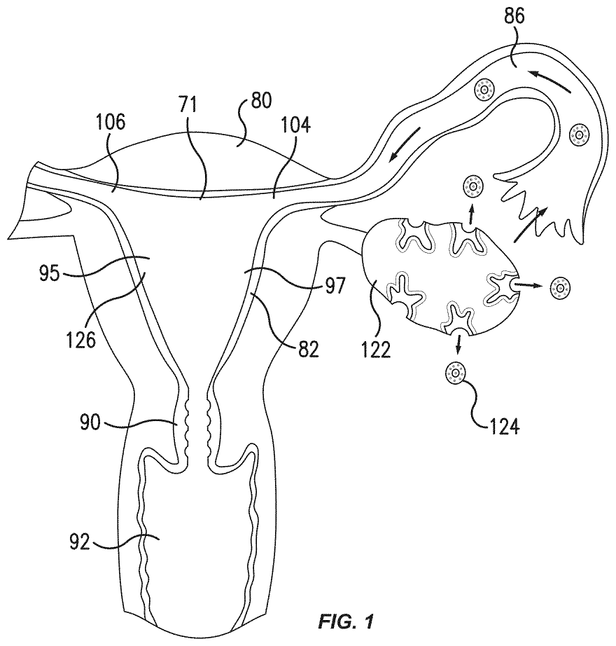

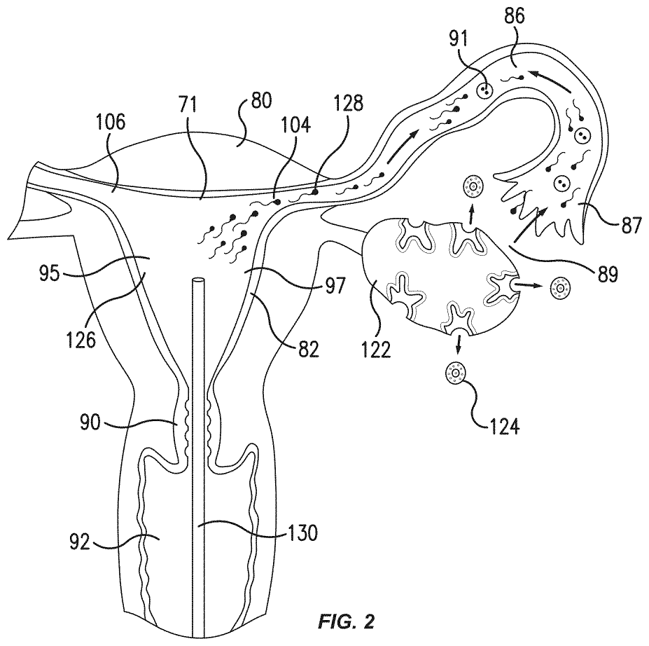

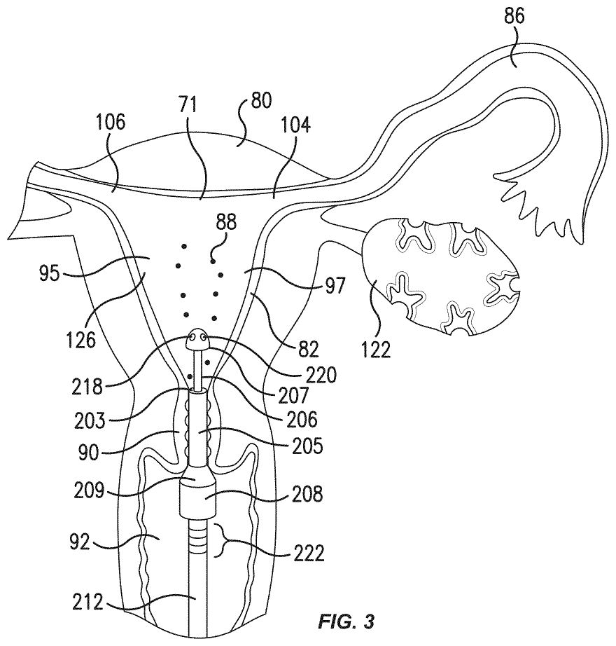

As shown in FIG. 1, in some embodiments of the technique that is described, an at-risk woman is induced to superovulate multiple oocytes 124 using fertility drugs. Superovulation is followed by intrauterine insemination by her partner's or donor sperm 128 and in vivo fertilization in her reproductive tract to produce preimplantation embryos 88 (blastocysts) (FIGS. 2-3). The blastocysts 88 (e.g., blastocysts of 5-8 days gestational age) are recovered by uterine lavage. Uterine lavage may be a nonsurgical office technique that allows recovery of human preimplantation embryos naturally conceived in vivo, in a woman's body.

In some embodiments, embryonic micromanipulation with biopsy then is used to remove trophectoderm (early placenta) or targeted inner cell mass (early fetal cells) from one or more of the recovered blastocysts. The biopsied trophectoderm cells are used, for example, for molecular diagnosis of specific genetic disorders. The diagnosis is followed by therapeutic embryonic intervention using selective replacement or gene therapy with specific corrective genetic constructs or stem cell/embryonic cell transplants. The diagnosed or treated embryos are then replaced into the woman's uterine cavity leading to a viable unaffected birth.

In some embodiments, the molecular diagnosis of an embryo can be performed without the need for a biopsy of the embryo. Uterine lavage recovered embryos can be placed into culture for up to 12 hours. During the culture period, the embryo extracts genetic material which can be analyzed. The genetic material secreted by the embryo is representative of the embryonic genome. The embryo extracts are used, for example, for molecular diagnosis of specific genetic disorders. The diagnosis is followed by therapeutic embryonic intervention using selective replacement or gene therapy with specific corrective genetic constructs or stem cell/embryonic cell transplants. The diagnosed or treated embryos are then replaced into the woman's uterine cavity leading to a viable unaffected birth.

Examples of genetic disorders that may be detected and/or corrected by gene therapy include, without limitation, 22q11.2 deletion syndrome, Angelman syndrome, Canavan disease, Charcot-Marie-Tooth disease, color blindness, Cri du chat, cystic fibrosis, Duchenne muscular dystrophy, familial hypercholesterolemia, haemochromatosis, hemophilia, Klinefelter syndrome, neurofibromatosis, phenylketonuria, polycystic kidney disease, Prader-Willi syndrome, sickle-cell disease, spinal muscular atrophy, Tay-Sachs disease and trisomy 21 (Down syndrome), and Turner syndrome.

In some examples of the approach that we describe here, uterine lavage, and ancillary devices, steps, and services related to it and built around it, provide a simple, safe, and inexpensive way to diagnose and treat human embryos before implantation (PGD, PGT-A, PGT-M or PGS) or to make a sex determination or both.

For convenience, we briefly discuss certain terms that we use in our description.

When we use the term superovulation, we intend to refer broadly to any production and release of many (for example, three or more) mature eggs 124 in one menstrual cycle, triggered, for example, by a medication that stimulates the ovaries.

When we use the term artificial insemination (AI), we include broadly any process by which sperm 128 is placed into the reproductive tract of a woman, for the purpose of impregnating her, by other than sexual intercourse. In some examples, the artificial insemination involves placing sperm, which has been processed by washing her partner's or donor semen, into the uterine cavity 126, and is sometimes called artificial intrauterine insemination (IUI), for example, as shown in FIG. 2. When IUI is combined with a sequence of injectable fertility drugs, there is an expected marked increase in pregnancy rates compared to insemination by sexual intercourse and spontaneous ovulation. Of course, in some embodiments, insemination by sexual intercourse and spontaneous ovulation, IUI and spontaneous ovulation, or insemination by sexual intercourse and superovulation are also contemplated.

We use the term in vivo fertilization broadly to include any fertilization within a woman's body, for example, the natural combination of an oocyte (egg) 124 and sperm 128 in the female reproductive tract that occurs as a result of sexual intercourse or after artificial insemination.

We use the term in vitro fertilization (IVF) to refer broadly to any fertilization that occurs outside of the woman's body, for example, when the oocyte and the sperm are combined in a laboratory dish. In some examples, the fertilized oocyte is incubated for 3 to 5 days in a chamber (incubator) that provides warmth and nutrients. After IVF, the embryo may be implanted into the uterus of a woman to carry the baby to term. IVF tends to be complex, inefficient, and expensive. Typically, the oocyte is recovered in an operating room under general anesthesia and is fertilized by injecting sperm (for example, ICSI: intracytoplasmic sperm injection) in a sophisticated laboratory facility. Live birth rates for PGT done by IVF normally run between 20 to 30% per treatment cycle; these rates are improving only modestly in recent years and are not expected to improve dramatically in the foreseeable future.

We use the term blastocyst to refer broadly to, for example, any human preimplantation embryo when it is in a developmental stage, for example, a stage that is typically reached at 4-5 days after fertilization and is observable in the uterus for up to 8 days after fertilization and just prior to implantation. A human blastocyst normally comprises of 100 to 300 cells and is a thin-walled embryonic structure that contains a partially differentiated cluster of cells called the inner cell mass from which the embryo arises. An outer layer of cells gives rise to the placenta and other supporting tissues needed for fetal development within the uterus, while the inner cell mass cells give rise to the tissues of the body. Located at the center of the blastocyst is a fluid-filled or gel-filled, hollow center or core called the blastocoel. The blastocoel core and the gel or fluid that comprises it comes into direct physical contact with the trophectoderm or inner cell mass cells that make up the blastocyst walls that surround that core. Human blastocysts, if removed from the woman, produce high singleton pregnancy rates when transferred back into the uterus and are considered to be at a good stage for preimplantation diagnosis, because there are many cells and a high likelihood of survival. In our discussion, the terms blastocyst and embryo are commonly used interchangeably.

When we refer to a catheter, we mean to refer broadly to, for example, any hollow tube that has any shape, form, weight, material, configuration, size, rigidity, durability, or other characteristics to be inserted into the uterus to permit fluid to pass to or from the uterus.

The term uterus refers to a hollow, muscular, pear-shaped organ, located in a pelvis of a woman between the bladder and the rectum where pregnancy implants, grows and is carried to viability.

We use the term cervix as shown, for example, as element 90 in the figures, to refer broadly to the lower, narrow segment of the uterus that embraces at its center an endocervical canal 157 connecting the uterine cavity with the vagina (see FIG. 10, for example). The cervix typically is dilated (that is, the canal is expanded or enlarged) to pass the instruments required for uterine lavage or for transfer of embryos back into the uterus.

We use the term fundus as shown, for example, as element 153 in FIG. 10 showing the top of the uterus.

We use the term uterine cavity broadly to describe the heart-shaped space shown, for example, as element 126 in an anterior-posterior view. Viewed as a lateral exposure, the uterine cavity 126 between the cervical canal and the fallopian tubes appears as a narrow slit. The uterine cavity space represents a potential space in the non-pregnant state, when the muscular front and rear (anterior and posterior) uterine walls are in direct contact with each other and separated only by a thin film of uterine fluid. The direct apposition of (contact between) the anterior and posterior walls of the uterine cavity 126 is apparent in a lateral view. Blastocysts and other preimplantation embryos are freely suspended in this film of intrauterine fluid before they implant into the wall of the uterus. The potential space becomes a real space when greatly expanded when, for example, the walls are separated mechanically by surgical instruments (such as catheters) or in the pregnant state when the pregnancy and its surrounding membranes separate the walls widely apart.

We use the term fallopian tube as shown, for example, as element 86 broadly to describe oviduct structures that enable, for example, transport of sperm cells from the uterus to the ovaries where fertilization takes place and for return transport of embryos back to the uterus for implantation.

Internal ostia refers broadly to openings in the uppermost uterine cavity that link and complete the passageway of the fallopian tubes from the ovaries to the uterus as shown, for example, as elements 104, 106.

The term internal os (or internal cervical ostium) refers to the opening of the cervix into the uterine cavity as shown, for example, as element 155.

The term external os (or external cervical ostium) refers to the opening of the cervix into the vagina as shown, for example, as element 170.

As we use the term, cryopreservation refers broadly to a process in which, for example, one or more cells, whole tissues, or preimplantation embryos are preserved by cooling to a temperature at which, for example, biological activity including biochemical reactions that would lead to cell death, are slowed significantly or stopped. The temperature could be a sub-zero ° C. temperature, for example, 77° K or −196° C. (the boiling point of liquid nitrogen). Human embryos can be cryopreserved and thawed with a high probability of viability after storage even of many years.

When we refer to intervention by embryo (gene) therapy, we intend to include broadly any strategy for altering a human physical condition, including, for example, treating a disease by placing (e.g., injecting) cells into an embryo, blastocyst or its blastocoele core, or placing (e.g., injecting) DNA (such as modified or reconstructed DNA) into individual embryonic cells or inner cell mass or trophectoderm cells or surrounding media so as to modify the genome of the embryo or blastocyst to correct, for example, a defective gene or genome.

In a general strategy, gene therapy at the embryonic blastocyst stage may involve replacing a defective gene of any genetic disease with an intact and normally functioning version of that gene. Replacement is performed by placing the replacement gene in the surrounding media or injecting the replacement gene by nanosurgical methods directly into the blastocoele of a blastocyst or selectively into its trophectoderm cells or inner cell mass.

In one strategy, the replacement gene or DNA sequence can be loaded onto a virus (for example retrovirus or adenovirus vector) which delivers the sequence into the trophectoderm cells or cells of the inner cell mass. Other intracellular delivery methods include use of other viruses and non-viral methods including naked DNA, chemical complexes of DNA or physical methods such as electroporation, sonoporation, or magnetofection.

The blastocyst is an excellent (perhaps ideal) site to implement gene therapy because the genetic constructs and viral vectors are likely not destroyed by the immunological response of an adult organism that may impair the success of gene therapy when applied to adults. Thus it is expected that incorporation of replacement genes and their viral vectors will be highly efficient at the blastocyst stage.

One example would be prevention or deletion or inactivation of the Hemophilia B gene in a human blastocyst Hemophilia B male carrier by injection of the replacement gene with an adenovirus vector into the surrounding media or blastocoel core allowing vector to contact and transfect virtually all trophectoderm and inner mass cells and be incorporated ultimately into all fetal and adult cells of the resulting newborn. Hemophilia B has been successfully treated in adult human subjects by gene therapy.

We use the term fertile couple to refer broadly to a man and a woman who have no known fertility disorders (for example, a biological inability of one of them to contribute to conception). Conversely, we use the term infertile couple to refer broadly to a man and a woman known to have a fertility disorder, for example a disorder in which unprotected sexual intercourse for over one year fails to achieve a viable pregnancy if the woman is 35 years old or less or six months of unprotected intercourse if 36 years old or older.

We use the term lavage fluid to refer broadly to any physiologic fluid that can be used in the process of recovering blastocysts from the uterus, for example, a wide variety of aqueous tissue-culture life-sustaining buffered salt solutions (media) (for example—Heapes based HTF with up to 20% protein) commonly used for oocyte aspiration, or used by embryology laboratories to sustain embryonic viability for long or short periods of time. Other common lavage fluids may include but are not limited to the following: Lactated Ringers, Global® Collect®, and Vitrolife® Media.

We use the term lavage fluid filtering broadly to refer to any kind of processing of uterine lavage fluid (for example, after it has been recovered from the uterus) to, for example, isolate human blastocysts from the fluid. Such filtering can include, for example, separating maternal intrauterine cells, mucous, and debris from the blastocysts.

We use the term preimplantation embryo to refer in a broad sense to, for example, an embryo that is free floating in a woman's reproductive tract after fertilization. A preimplantation embryo can have, for example, one cell with a male and female pronuclear (day 0) graduating to two cells (day 1) to 2-4 cells (on day 2) to 6-10 cells (day 3), to blastocysts (day 5 to 8) with 100 to 300 cells. Typically, a pregnancy is established when a preimplantation embryo implants into the uterine wall on day 7 or 8 and begins to interact with the maternal blood supply.

We use the term oocyte to refer in a broad sense, for example, an unfertilized egg that has been retrieved, or is released naturally, from the ovaries.

We use the phrases preimplantation genetic diagnosis (PGD) or preimplantation genetic testing for monogenic/single gene disorder (PGT-M) broadly to refer, for example, to any kind of genetic diagnosis of embryos prior to implantation. PGD or PGT-M can, for example, reduce the need for selective pregnancy termination based on pre natal diagnosis as the method makes it highly likely that the baby will be free of the disease under consideration. In the current practice, PGD or PGT-M uses in vitro fertilization to obtain oocytes or embryos for evaluation.

We use the phrase pre-implantation genetic screening (PGS) or pre-implantation genetic testing for aneuploidy (PGT-A) broadly to denote, for example, procedures that do not look for a specific disease but to determine the presence of a normal number of chromosomes using genetic techniques that can determine if an excess or deficiency of chromosomes are observed in addition to the genetic sex of the embryo. So both PGD and PGS may be referred to as types of embryo screening.

We use the phrase pre-implantation genetic testing for structural rearrangements (PGT-SR) broadly to denote, for example, procedures that will determine whether an embryo has a chromosomal rearrangement such as an inversion, reciprocal translocation, or Robertsonian translocation. PGT-SR can, for example, reduce the need for selective pregnancy termination based on pre natal diagnosis as the method makes it highly likely that the baby will be free of chromosomal rearrangements under consideration. In the current practice, PGT-SR uses in vitro fertilization to obtain oocytes or embryos for evaluation. So PGT-A, PGT-M, and PGT-SR may be referred to as types of embryo screening.

When we use the term uterine lavage, we intend to refer broadly to any possible lavage technique for recovery of one or more oocytes (e.g., unfertilized eggs) or human embryos (e.g., blastocysts). Oocytes are recovered within 24 hours of the time of ovulation. Embryos are recovered from a living healthy woman after formation of the embryos, for example, before the embryos have established a pregnancy by attachment to the uterus. In some examples, the lavage includes flushing fluid, for example, cell culture fluid, into the uterus and capturing the flushed fluid from the uterus to recover the oocytes or blastocysts.

When we use the term recovery in reference to oocytes and blastocysts, we intend to include broadly any process of any kind, form, duration, location, frequency, complexity, simplicity, or other characteristic that is used to retrieve one or more oocytes and blastocysts from a woman.

The term recovery efficiency within a given cycle refers broadly to, for example, the number of oocytes and embryos recovered (e.g., by uterine lavage) from a woman expressed as a percentage of the total number of follicles greater than or equal to 16 mm in diameter at the time the superovulation cycle is triggered. The number of follicles is determined through ultrasound evaluation of the ovaries. The catheter is designed to maximize recovery efficiency. Recovery efficiency is a direct reflection of the safety of the instrument. Lower recovery efficiency may increase the likelihood of residual embryos being present in the woman after the lavage procedure. Any embryos present after the lavage are by definition pregnancies of unknown location (PUL).

The term recovery frequency refers broadly to, for example, the number of uterine lavage cycles that produce at least one oocyte or embryo per every 10 individual cycles. For example, if in 5/10 cycles the catheter recovers at least one oocyte or embryo, the recovery frequency would be 50%.

Women with normal reproductive efficiency are expected to ovulate between 1 to 20 oocytes (e.g. unfertilized eggs) following any superovulation cycle. Of those eggs, between 50-100% will be successfully fertilized with donor or partner sperm and produce an embryo. At least 1 embryo is expected per superovulation cycle, this number may vary widely depending on the patient indication. The expected recovery efficiency for those embryos is at least 95%-100%, or in some cases at least 95% or in some cases at least 90% or in some cases at least 80% or in some cases at least 50%. Recovery efficiency is expected to decrease with advancing maternal age, and applying the techniques described here for more than one ovulation cycle is expected to be required for older women or women with borderline fertility.

It may be desirable to adjust the parameters and approach to the procedures that we have described here to achieve the greatest possible recovery efficiency. Achieving a high recovery efficiency is both advantageous to the woman because it implies that fewer blastocysts will remain in the uterus that could potentially implant. High recovery efficiency is also desirable because it will improve the statistical likelihood that, among the blastocysts recovered, one or more will be suitable for treatment (or will not need treatment) and can be transferred into the woman, without requiring repetitions of the procedure. In this sense, higher recovery efficiency will also mean lower cost.

As we have described here, appropriate treatments delivered to the woman at the appropriate times can reduce or eliminate the chance of any unintended implantation of a blastocyst that has not been recovered during the lavage.

Achieving 100% recovery efficiency is the goal of the procedure, but any recovery efficiency of 50% or more is expected to be desirable and useful. Commercial viability of the procedure is expected to be good if the recovery efficiency can be at least 80% or at least 90%. Recovery efficiency of at least 95% should provide excellent commercial feasibility possibilities.

The terms GnRH (gonadotropin releasing hormone) antagonist or agonist are used broadly to refer, for example, to a class of modified central nervous system hormones that are used as injectable drugs to stimulate or shut down release of pituitary hormones (e.g., FSH) that regulate human ovulation and release of ovarian hormones.

The term FSH (follicle stimulating hormone) refers to a pituitary hormone that naturally regulates the maturation and release of ovarian follicles and oocytes. Injected as a therapeutic agent, FSH can stimulate the maturation of multiple oocytes.

The term LH (luteinizing hormone) refers to a pituitary hormone that naturally induces the release of oocytes at ovulation. Injected as therapeutic agent, LH (or various surrogates) can induce release of oocytes at ovulation at a time determined by the time of injection.

The process of uterine lavage may include, for example, a series from superovulation to embryo recovery, embryo management, and transfer of selected or treated in vivo embryos.

Superovulation is induced using injectable FSH to stimulate maturation of multiple oocytes. GnRH agonists are combined with the FSH and their purpose is to quiet the ovaries into a pseudo-menopause state and prevent premature ovulation in the superovulation cycle. GnRH antagonists are also used to prevent premature ovulation in the superovulation cycle. Injectable hCG, or LH, or an LH surrogate (GnRH agonist that stimulates the pituitary to secrete natural LH) is then used to complete (trigger) the superovulation process (the release of multiple unfertilized oocytes 124 from both of the ovaries 122). In some implementations, one or more of these steps used for in vivo fertilization are similar to, but not exactly the same as, those used to induce superovulation by fertility clinics for IVF cycles. For in vivo fertilization, standard IVF superovulation methods, for example, are modified to reduce risks of ovarian hyperstimulation and retained pregnancies resulting from blastocysts not recovered in the uterine lavage.

In some implementations, the modifications include that the superovulation cycles use GnRH antagonists (GnRH receptor blocker peptides such as Cetrotide 0.25 or 3 mg, Ganirelix, Abarelix, Cetrorelix, or Degarelix) to prevent premature ovulation during stimulation with FSH. The FSH stimulates maturation of multiple oocytes. In some instances, FSH is self-injected using daily doses of FSH (Duration of use: 5 to 15 days given at ranges of 12.5 to 600 IU per day). Gonadotropin preparations may include injectable preparations containing FSH and LH, purified FSH (urofollitropins), or recombinant FSH, or single doses of long acting recombinant FSH.

In some implementations, the oocytes are released (triggered) by a single subcutaneous dose (e.g. 0.5 mg) of a GnRH agonist. Commonly used GnRH agonists include: Leuprolide acetate or Nafarelin Acetate or Buserelin. GnRH agonists (which release endogenous LH) may either be injected or administered via the intransal route. The GnRH agonist trigger minimizes risk of hyperstimulation due to the short half-life of the released LH hormone.

In some implementations, a GnRH agonist may be combined with hCG or injectable recombinant LH to complete (trigger) the superovulation process,

Approximately 24-36 hours after the ovulation trigger, an artificial insemination procedure is performed using partner or donor sperm. Artificial insemination techniques include intracervical insemination or intrauterine insemination (IUI). Following insemination, the multiple eggs released from the ovaries are expected to be fertilized in the fallopian tubes and migrate to the uterus as they continue embryogenesis. The point of insemination is the reference point for the lavage procedure, which is scheduled at approximately between 3 and 7 days after insemination.

In a uterine lavage cycle for oocyte recovery, approximately 24-36 hours after the ovulation trigger, the multiple eggs released from the ovaries are expected to be transported from the fallopian tubes into the uterus. No insemination is performed. the lavage procedure described herein may be performed within 0 to 36 hours of natural ovulation (or within 0 to 24 hours, 12 to 36 hours, or 24 to 36 hours of natural ovulation) to recover oocytes that are present in the uterus. In various embodiments, the lavage procedure described herein may be performed within 36 to 72 hours after the application of an ovulatory trigger (or within 36 to 60 hours, 48 to 72 hours, or 48 to 60 hours after application of an ovulatory trigger).

In some implementations, because there is risk of corpus luteum apoptosis (collapse) with antagonist suppressed cycles, progesterone (given as vaginal progesterone, Crinone® 8%, 1 dose per day or Prometrium® 200 mg 3 capsules placed within the vagina per day) or oral progesterone (or Prometrium® 200 mg 3 oral capsules per day) and oral or transdermal estradiol (transdermal estradiol patches 0.1 mg per day or oral estradiol 4.0 mg per day) are administered until the day of lavage.

In some implementations, after lavage, both progesterone and estradiol are discontinued. Uterine lavage is performed between days 3 and 7 days after insemination and the embryos are recovered. At the end of the lavage, before or shortly after removal of the catheters, a single dose of progesterone receptor antagonist (Mifepristone 600 mg) is injected into the uterine cavity with a second dose (Mifepristone 600 mg) mg given by mouth one day prior to expected menses.

In some implementations, after lavage, GnRH antagonist is administered (e.g. Cetrotide 3 mg) on the day of lavage recovery to induce corpus luteum apoptosis and suppress luteal phase progesterone and decrease further risk of a retained (on account of blastocysts missed by the intrauterine lavage) pregnancy. GnRH antagonist administration starts before or on the day of lavage recovery and may continue daily utilizing dosages of about 0.25 to 10 mg for up to 10 days following lavage or until menses, whichever occurs first. This novel use of a GnRH antagonist for corpus luteum suppression following blastocyst recovery after superovulation reduces or eliminates the possibility that unrecovered blastocysts will implant and result in unintended pregnancy. Uterine lavage done on non-stimulated cycles has a significantly lower risk of retained and/or ectopic pregnancy.

As explained, because the superovulation and artificial insemination produce viable multiple blastocysts within the uterus, and because the lavage may possibly not recover all of the blastocysts from the uterus, it is important to take steps, such as those mentioned above, to reduce or eliminate the possibility that unrecovered blastocysts will implant and result in unintended pregnancy. Additionally, although examples of protocols for achieving superovulation and steps that follow it are described above, a variety of other protocols may be safe and effective. Other protocols may be able to achieve the functions and results mentioned. For example, other regimes may be possible to quiet the ovaries into a pseudo-menopausal state, to trigger maturation of multiple oocytes, to stimulate superovulation, to minimize the risk of overstimulation, to reduce the risk of corpus luteum collapse, and in general to reduce the risk of an unintended retained pregnancy.

Again with reference to FIGS. 1-2, the released oocytes 124 are captured in the open end of the fallopian tube 86 and move towards the uterine cavity 126 naturally after ovulation. The oocytes 124 are fertilized in the woman's fallopian tubes 86 or in the area 89 of the peritubal-ovarian interface adjacent to the ovary where the tubes open in contact with or in close approximation to the ovary.

As shown in FIG. 2, artificial intrauterine insemination (IUI) is performed using a commercially available intrauterine insemination catheter 130 to inject washed semen 128 through the vagina 92 and cervix 90 directly into the uterine cavity 126 one or more times per superovulatory cycle. IUI is performed after superovulation, approximately 24-40 hours after triggering this event with the GnRH agonist and/or hCG or LH surrogate. This IUI procedure delivers sperm 128 cells into the uterus that then become available in very large numbers for in vivo fertilization. Of course, timed intercourse is also possible.

As shown in figures, in vivo fertilization occurs by natural means after artificial insemination with washed semen 128. The sperm cells 128 migrate to and through the internal ostia 104, 106, into the oviducts 86 migrating to the distal oviduct 87 into the peritubal-ovarian interface 89 in contact with and adjacent to the ovary 122 where sperm cells contact and interact with the released oocytes 124 to fertilize these oocytes 124 in vivo.

Typically the sperm 128 travel up the fallopian tube toward and fertilize the oocytes, which then become embryos. The embryos 88 (see, e.g., FIG. 3) then continue to move toward and into the uterine cavity 126 where by the fourth to sixth day they mature to blastocysts 88 and are free floating in a thin film of uterine fluid between the anterior and posterior surfaces of the middle uterine cavity.

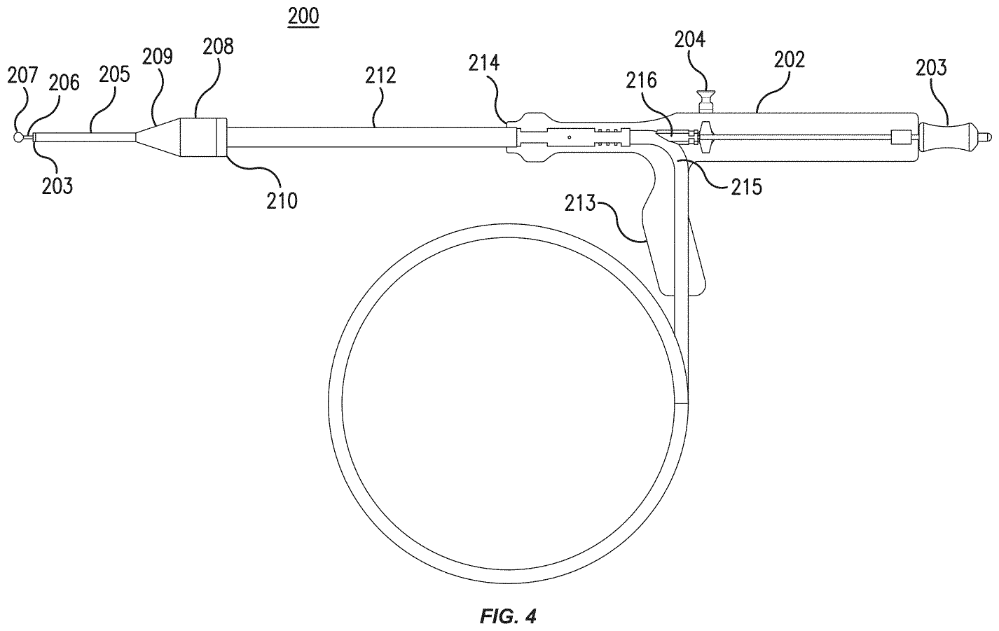

Turning to FIGS. 3 and 4, a portion of a uterine lavage catheter device 200 is shown, showing a portion deployed transvaginally within a human uterus, in the process of recovering blastocysts 88. As shown in FIGS. 3 and 4, for example, uterine lavage catheter device 200 comprises a seal element 208 configured to provide a sealing surface 209 against the exterior cervical ostium. In some embodiments, uterine lavage catheter device includes a supply lumen 206, which extends from seal element 209 and is configured to supply lavage fluid as described above. A nozzle 207 is configured to generate a spray pattern of lavage fluid and is coupled (e.g., fluidically) to supply lumen 206. In some embodiments nozzle 207 may be integral with supply lumen 206. Additionally, uterine lavage catheter device 200 includes return lumen 205, in some embodiments. Return lumen 205 is configured to retrieve lavage fluid and cells from the uterus, for example blastocysts 88.

Uterine lavage catheter device 200 may include index markers 222, noting position of seal element 208 relative to extension tubing 212 (which controls the length of return lumen 205 and positioning at internal cervical ostium). In some embodiments, extension tubing 212 may house both return lumen 205 and supply lumen 206. In some embodiments, extension tubing 212 may be configured as either return lumen 205 or supply lumen 206. Index markers may be referred to as a cervical stop scale, and are etched into the outside of the return lumen 206 in some embodiments. Index markers 222 mark the position of the sealing surface when it is custom-adjusted to each patient prior to insertion.

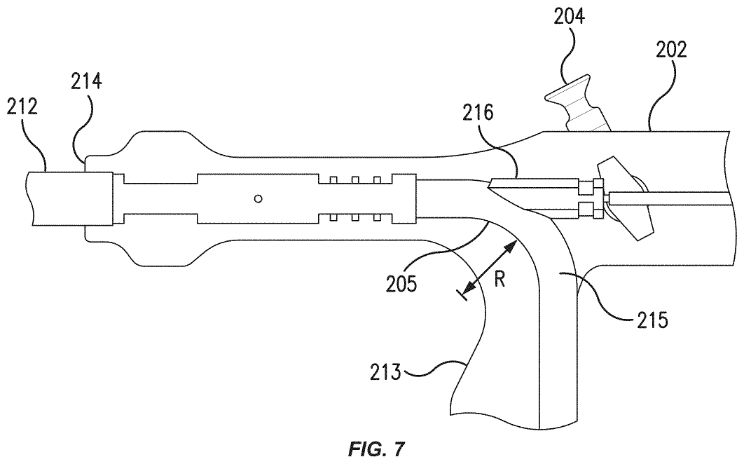

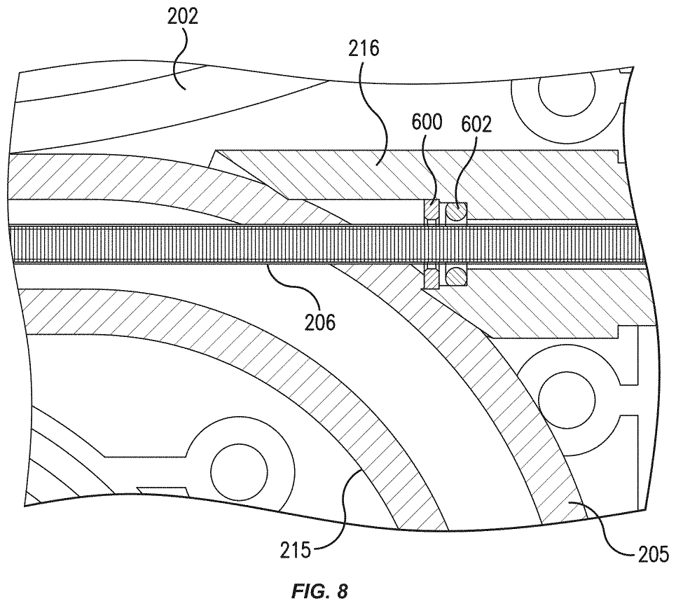

FIG. 4 shows a simplified cross sectional view of uterine lavage catheter device 200. As shown, catheter device 200 includes housing 202, which houses certain steerability components, the fluid manifold, and other fluidic connections. Housing 202 may include proximal end 214, from which extension tubing 212 extends. Housing 202 is shown to include a handle 213, which may be used by a physician during a lavage procedure. Translation control element 203 is coupled to supply lumen 206, and controls the translation of the supply lumen along the longitudinal axis of catheter device 200. As shown, a simplified manifold is provided, including seal housing 216 at a point where the supply lumen 206 exits return lumen 205. The manifold may include transition portion 215 which may include a radius of curvature, further described with reference to FIGS. 7 and 8, below.

As shown in FIGS. 3, 4, and 5, for example, seal element 208 includes the distal surface 209, which may function as a sealing surface. Return lumen 205 includes an inlet 203, and return lumen 205 may be disposed coaxial the supply lumen 206. In some embodiments, return lumen 205 may house supply lumen 206. In some embodiments inlet 203 of return lumen 205 is located a first distance X₃ from sealing surface 209, such that the inlet 203 of return lumen 205 is configured to be placed at the interior cervical ostium. In some embodiments, seal element 208 comprises a conical distal surface 209, the comprises a sealing surface. Seal element 208 may further include proximal end 210 which may connect to another portion 212 of the main catheter device body.

With return lumen 205 and supply lumen 206 in place under ultrasound guidance, the operator (for example, a physician, specially trained technician, or nurse) inserts and steers the body of catheter device 200 into the uterine cavity. The catheter tip is echogenic such that it may be visualized clearly under ultrasound. Either vaginal and/or abdominal ultrasound may be utilized to properly visualize the catheter within the uterine cavity. A lavage controller connected to the system is prompted to begin the lavage with preset delivery frequencies of lavage fluid and collection of embryos into return lumen 205.

The lavage cycle is started when the controller is prompted to begin the preset lavage cycle of pulsed fluid delivery with continuous collection. The first stage of the lavage cycle is begun by injecting a small amount of fluid into the uterine cavity to form a puddle of fluid encompassing the pre-implantation embryos, e.g, blastocysts 88. A fluid flow with one or more entrained pre-implantation embryos or oocytes continuously travels to a collection vessel. The second stage of the lavage cycle is begun by injecting pulsatile fluid into the uterus to keep fluid in motion out of the uterus. At the conclusion of the cycle, all of the fluid present in the uterine cavity is then returned from the catheter device and tubing along with one or more entrained embryos or oocytes into the collection vessel.

In some embodiments, the controller may include a display configured to display a graphical user interface (GUI) showing patient information (not shown), a type of instrument, a type of fluid dosage, a selected volume of fluid dosage, a fluid flow rate 1408, a time status of operation, a remaining volume of fluid dosage, and operation controls. The GUI may be used to control various parameters of the lavage cycle. In some embodiments, the controller may include an RFID interface. The RFID interface may communicate with the lavage fluid bottle and/or catheter. In this way, information such as serial numbers, lot numbers, etc., are recorded by the controller, and may be used to unlock the controller (e.g., log-in to the interface and allow the system to be used). In some embodiments, the RFID interface may prevent users from re-using catheters, prevent users from using non-compatible consumables such as the lavage fluid bottle, etc.

The uterine lavage procedure is performed under low flow conditions, as managed by the controller, not to exceed the maximum pressure allowed by the device of between about 25 mmHg and about 75 mmHg (or between about 10 mmHG and about 105 mmHg) of intrauterine pressure to maintain the expansion of the uterus during fluid delivery and removal. In other embodiments, the controller may set the maximum pressure to be between 20 PSI and 80 PSI. In some embodiments, the controller may set the maximum pressure to be less than about 80 PSI, less than about 60 PSI, less than about 50 PSI, or less than about 40 PSI, for example. The uterine cavity is slightly expanded. The lavage device does not include any members that act to expand the uterine cavity. The lavage process was designed to prevent the introduction of air into the uterine cavity to ensure the health and integrity of the recovered blastocysts, or to prevent the risk of air embolism. In some embodiments, the controller is programmed to both deliver lavage liquid to the uterus and apply vacuum in a pulse that alternates suction and pulses cadenced exactly the opposite fluid delivery at a preset frequency of, for example, between about every 0.5 to about every 4.0 seconds, or between about every 0.25 to about every 8.0 seconds. In some embodiments, the controller is programmed to both deliver lavage liquid to the uterus and apply a pulsed cadenced at a preset frequency which, which does not allow the fluid velocity to reach zero for example, between about every 0.5 to about every 4.0 seconds, or between about every 0.25 to about every 8.0 seconds. Uterine lavage fluid is delivered into the uterus at a low flow of fluid supply that does not exceed a maximum flow out of the device between about 100 mL per minute through about 250 mL per minute (between about 50 mL per minute through about 350 mL per minute) through the outlets of nozzle 207. Lavage fluid is delivered in short pulses through the ball tip with highly focused stream of fluid directed to the uterine cavity wall near the fundus 153/71 with a turbulent action to produce effective lavage. Intermittent pulsatile flow through nozzle 207 allows for orderly movement of fluid containing embryos through return lumen 205 to the embryo recovery vessel. By the combination of direct stream-forcing embryos away from the fundus 153/71 combined with continuous flow through the return lumen placement there should be no lavage fluid or embryonic losses. Thus, this arrangement and other features of the instruments and procedure are designed to achieve the ideal goal of removing all of the oocytes or embryos present in the uterus through the return line, to leave none of them in the uterus, and to force none of them into the fallopian tubes.

One approach for ensuring constant flow is storing the fluid pressure energy in the supply tubing of the catheter device. A hydraulic accumulator may be used to store the fluid pressure energy in the supply tubing. An accumulator typically includes a discrete device comprising a chamber with an elastic element, such as a piston, diaphragm or spring. However, in the various embodiments of the present disclosure, the elasticity of the supply tube is tuned by pulses to store fluid pressure such that fluid flow is maintained when the pulsatile action of the lavage pump ceases. The use of pulsatile fluid flow, as described herein, simplifies the pump control by using precisely timed on/off cycles. In other embodiments, the pump motor may be slowed by a precise amount without coming to a complete stop.

In some embodiments, the collection system comprises a collection bottle disposed external to catheter device 200 and can be raised or lowered to manage intrauterine pressure. The collection bottle comprises a collection mechanism for receiving fluid from the uterus and defines a fluid head to control intrauterine pressure and uterine expansion.

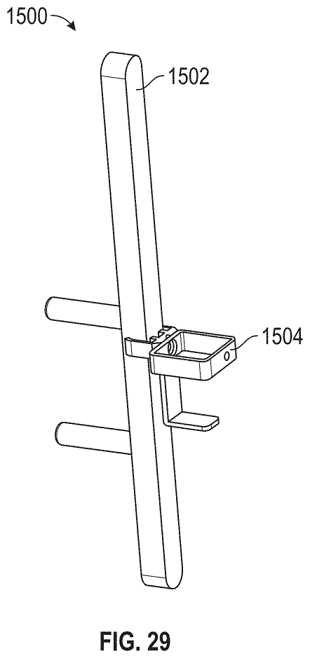

As shown in FIG. 29, the collection system may comprise an elevator 1500 configured to raise or lower the collection bottle to adjust intrauterine pressure. In the illustrated embodiment, elevator 1500 comprises a track 1502 and a cradle 1504 coupled to the track 1502. In various embodiments, cradle 1504 is configured to receive and hold the collection bottle. In some embodiments, cradle 1504 comprises a magnet for magnetically coupling cradle 1504 to the collection bottle or a pin for engaging the collection bottle.

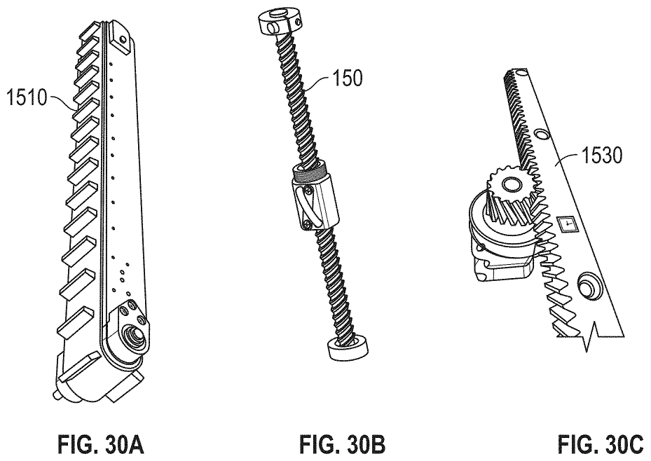

In various embodiments, elevator 1500 is configured to raise or lower cradle 1504 along track 1502 to adjust fluid head of fluid stored in the collection bottle. In one embodiment shown in FIG. 30A, elevator 1500 comprises a toothed belt assembly 1510 configured to raise and lower cradle 1504 along track 1502. In one embodiment shown in FIG. 30B, elevator 1500 comprises a screw-drive assembly 1520 configured to raise and lower cradle 1504 along track 1502. In one embodiment shown in FIG. 30C, elevator 1500 comprises a rack-gear assembly 1530 configured to raise and lower cradle 1504 along track 1502. In other embodiments (not shown), elevator 1500 may comprise other lifting mechanisms, such as a hydraulic assembly, to raise and lower cradle 1504.

As shown in FIGS. 3-5, for example, in some embodiments supply lumen 206 is coaxial with return lumen 205. In this way, the crossing profile, e.g., transverse footprint of catheter device 200 is optimized.

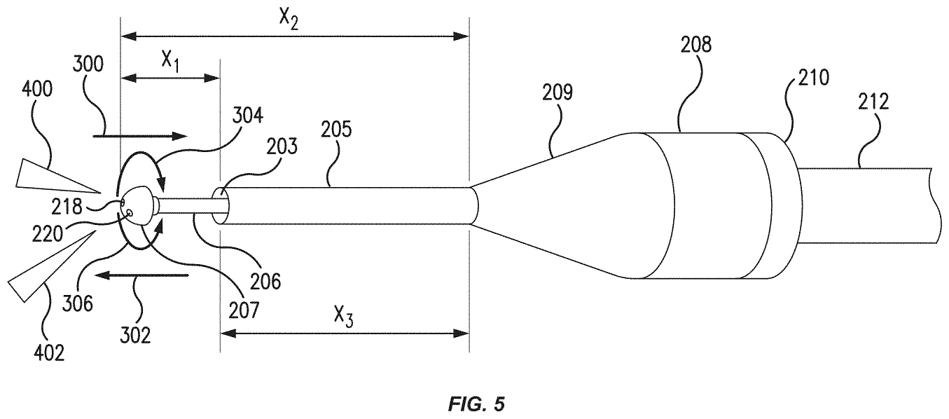

In some embodiments, nozzle 207 is configured to generate an asymmetric spray pattern. In some embodiments, nozzle 207 by way of supply lumen 206 is translatable along an axis corresponding to a linear, longitudinal axis of supply lumen 206 and by extension return lumen 205. Thus, a spray pattern, whether asymmetric or not, may travel further into the uterine cavity to provide lavage to the uterine walls. In some embodiments, nozzle 207 by way of supply lumen 206 is rotatable about the axis corresponding to a linear, longitudinal axis of supply lumen 206 and by extension return lumen 205. In some embodiments, nozzle 207 is both translatable along and rotatable about the same axis. As shown in FIG. 5, forward translation is indicated by element 302, backward translation is indicated by element 300, positive rotation is indicated by element 306, and negative rotation indicated by element 304. In some embodiments, nozzle 207 includes a first fluid outlet 218, and a second fluid outlet 220. First fluid outlet 218 and second fluid outlet 220 may be angularly offset from one another with respect to the axis of the supply lumen 206. The angle between first fluid outlet and second fluid outlet 218/220 may be between about 0 degrees and 60 degrees. In some embodiments, one of the first or second fluid outlet may be disposed between about 5° and 25° off-axis. In some embodiments, the other of the first or second fluid outlet is disposed between about 25° and 55° off axis. In some embodiments the first level in the second fluid outlet may be linearly offset from one another with respect to the axis of the supply lumen 206. First fluid outlet 218 may differ in size or shape from second fluid outlet 220.

Nozzle 207 may be, for example, a hollow ball fabricated from polymer, high-grade steel, or composite. The outlets may be internally tapered. The lavage fluid streams contact, break up, flush, and force mucous and cellular debris from the uterus into the wide, return lumen 205. The ports may be configured to deliver higher pressure, higher flow, and highly focused stream. The configurations of the ports may be customized individually in accordance with the uterine anatomy of a particular patient, determined at trial lavage. The angle between the directions of the two streams will as required to direct the fluid stream toward the fundus structures. After the angle is determined, catheters are supplied and customized for that one patient based on earlier measurements. The catheters may be disposable.

In some implementations, the supply lumen 206 may include secondary outlets (e.g., low pressure outlets/ports that direct streams of lavage fluid into the center of the uterine cavity 126 and downward into the return lumen 205). The flow of the lower pressure streams is restricted to the middle parts of the uterine cavity, are less forceful and less directed than the flows from the other outlets. The purpose of the lower pressure streams is to provide a diffuse pool of fluid that will solubilize the mucous matrix of the intrauterine fluid and facilitate a sweeping current containing all embryos in the uterus and facilitate their direction into return lumen 205.

In some embodiments, additional fluid outlets are contemplated. The fluid outlets may be symmetrical or asymmetrical. Fluid outlets may also be disposed symmetrically or asymmetrically about nozzle 207. Fluid outlets may be of any shape, however in some embodiments each may comprise a circular hole, for example approximately 0.025 inches in diameter. In some embodiments, nozzle 207 is generally formed as a hemisphere. In some embodiments (see FIG. 13), nozzle 207 may include proximal surface 221 which may be configured to enter return lumen 205 when supply lumen 206 is retracted into return lumen 205 such that return lumen 205 is in an aligned configuration. Proximal surface 221 may be conical for example, and may be formed integrally with the rest of nozzle 207. Proximal alignment surface 221 may be configured to align the inlet of suctioned lumen 205 when the supply lumen 206 is in a first position. In some embodiments proximal alignment surface 221 and extends into the inlet of the return lumen 205 when the supply lumen 206 is in the same first position.

The described configurations above, enable users to lavage larger areas of the uterus that be reached if the ports were arranged in other configurations. Combined with the rotation/translation of nozzle 207, a fluid flow is created by spray paths 400 and 402 (shown schematically in FIG. 5). Spray paths 400/402 create a fluid flow effect of two concentric circles, optimizing reach of areas lavaged in the uterus. In these dual concentric lavage zones, one radius of fluid spray is within the second. The configurations described provide more thorough lavage coverage within the uterus.

As described, nozzle 207/supply lumen 206 may be translated longitudinally to further increase the lavage zone, from the internal cervical ostium to the fundus. Translation of nozzle 207/supply lumen 206 is additionally advantageous as it improves the patency of the central collection line by dislodging any cells that may obstruct the exit port of the catheter.

With reference to FIGS. 3-6, a collection system is described. As discussed, the uterine lavage catheter device 200 includes return lumen 205, which may be a single fluid return port on the catheter device 200. During use, the inlet to return lumen 205 is placed at the internal cervical ostium 155 (see FIG. 10). In this way, return lumen 205 is configured as a central collection port and exit point for all fluid (e.g., lavage fluid containing blastocysts 88) meeting the uterus and leading to a single return collection tube. A single collection point positioned at the internal cervical ostium is advantageous compared to plural collection points, as multiple collection points divide return flow and decrease fluid collection velocity. This adversely impacts collection efficiency. Moreover, a single central collection point reduces stagnation caused by multiple collection points, e.g., zones on the surface of the catheter. Stagnation zones are areas where cells (e.g., oocytes, blastocysts, etc.) become stuck due to a zero velocity flow and are unable to be collected. For example there exists zero velocity flow at a point in between multiple collection points which results in cells parking, i.e. stagnation. Configuring return lumen 205 as a single central collection point increases embryo collection efficiency. By locating the central collection point such that it is a aligned with the internal cervical ostium, such a design may avoid a “standpipe” in the uterus.

As shown in FIG. 5, various distances may be specified to achieve the positioning as described above. As discussed, some embodiments inlet 203 of return lumen 205 is located a distance X3 from sealing surface 209, such that inlet 203 of return lumen 205 is configured to be placed at the interior cervical ostium. In some embodiments, X3 may be between about 3 cm and about 7 cm (or between about 1 cm and about 10 cm). In some embodiments, X3 may be about 5 cm. In some embodiments, catheter device 200 defines a distance X1 between the distal end of nozzle 207 and inlet 203 of return lumen 205. In some embodiments, X1 may be between about 1 cm and about 5 cm (or between about 0.5 cm to about 10 cm). In some embodiments, X1 may be about 3 cm. In some embodiments supply lumen 206 is translatable along shared lumen axis between about 1 mm and 5 mm. Catheter device 200 may define a distance X2 between the sealing surface 209 and end of nozzle 207. In some embodiments, return lumen 205 is the sole suction path fluid supplied by supply lumen 206 and blastocysts 88. In some embodiments, X2 may be between about 6 cm and about 12 cm (or between about 3 cm and 15 cm). In some embodiments, X2 may be about 8 cm.

In some embodiments, catheter device 200 may include an automated translation system configured to monitor the position of nozzle 207 and adjust the position of the nozzle 207 relative to the inlet 203 of return lumen 205. In some embodiments, the automated translation system comprises a proximity sensor connected to the nozzle 207, a translator operatively connected to supply lumen 206, and a nozzle control module (e.g., computer-medium readable instructions) stored in a memory of the lavage controller. In various embodiments, the translator is configured to translate supply lumen 206 in a longitudinal directly along the common lumen axis of catheter device 200 to adjust the position of nozzle 207 relative to inlet 203 of return lumen 205. In some embodiments, the translator comprises an electric-servo motor (e.g., step motor) or a hydraulic assembly to move supply lumen 206. In some embodiments, the translator is configured to use the lavage fluid pump itself as a motive mechanism.

In some embodiments, the proximity sensor is configured to detect the presence of a target surface (e.g., a surface along fundus 153) and transmit a signal indicating the presence of the target surface. In some embodiments, the proximity sensor is a pressure sensor configured to measure intrauterine pressure during the lavage procedure 157 and transmit a signal indicating the pressure of the uterus during the lavage. In some embodiments, the proximity sensor is an echo sensor configured to generate a sound wave and measure the velocity of the sound waves rebounding from fundus 157.

In some embodiments, once the lavage controller receives the signal transmitted by the proximity sensor, the nozzle control module is configured to instruct the lavage controller to determine a location of nozzle 207 relative to the target surface based on the received signal transmitted from the proximity sensor. In some embodiments, the nozzle control module is configured to instruct the lavage controller to actuate the translator to translate supply lumen 206 so that nozzle 207 moves closer or further away from target surface.

In some embodiments, the nozzle control module sets a pressure threshold and instructs the lavage controller to calculate a difference between a measured pressure from the proximity sensor and the pressure threshold. In some embodiments, the nozzle control module instructs the lavage controller to actuate the translator to adjust position of nozzle 207 based on calculated difference in pressure.

In some embodiments, the nozzle control module sets a sound wave velocity threshold and instructs the lavage controller to calculate a difference between a measured velocity from the proximity sensor and the velocity threshold to determine a position of nozzle 207. In some embodiments, the nozzle control module instructs the lavage controller to adjust position of nozzle 207 based on calculated difference in wave velocity.

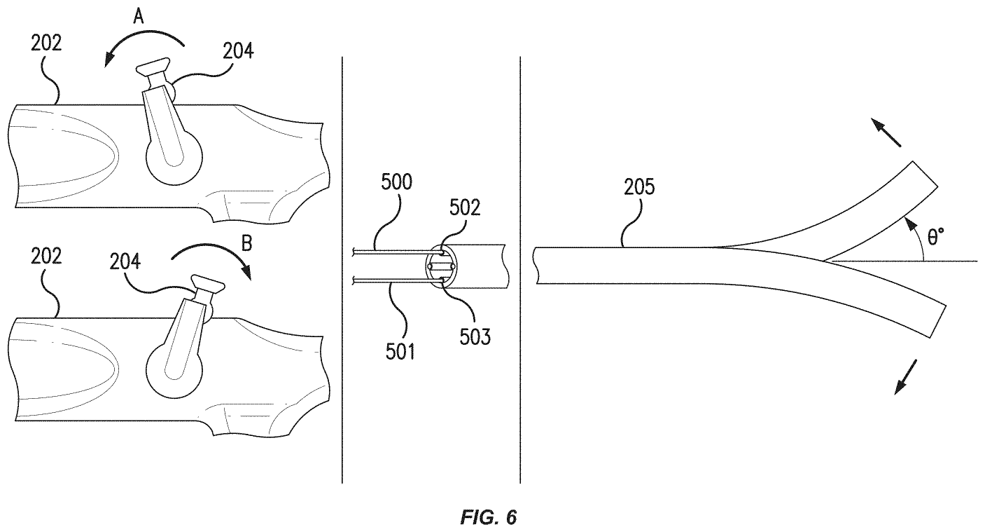

As shown in FIG. 6, for example, in some embodiments, return lumen 205 is steerable between about −60 degrees and about +60 degrees off-axis (or between about −90 and about +90 degrees off axis), however in some embodiments steerability is not provided for. Moving from left to right, steering element 204 is shown in a first position A, rotated about an axis pivot point of catheter device housing 202. In first position A, return lumen 205 may be steered upwards towards positive 0 degrees. Steering element 204 is coupled to guide wires 500 and 501 which are embedded in the wall of return lumen 205, for example, within channels 502 and 503, respectively. In response to steering element 204 for being rotated the opposite direction towards position B, return lumen 205 may be steered downwards, that is away from positive θ degrees. In some embodiments, return lumen 205 may be steerable between about 45° in either direction. By allowing return lumen 205 to be steerable, additional degree of catheter movement laterally is provided. In this way, catheter tip steerability provide easier access through tortuous/complex cervical anatomies, and enables users to maintain central positioning between anterior and posterior walls of the uterus. In some embodiments, this may be especially useful for anteflexed or retroflexed uteri. In this way catheter device 200 may operate in these difficult uterine anatomies and maintain central positioning of return lumen 205, thereby increasing collection efficiency of blastocysts 88. Moreover, this type of steerability facilitates the procedure and decreases the skill level required. It reduces manipulation and force necessary to gain access into the uterus, and improves patient comfort. In some embodiments, such steerability may eliminate the need for a tenaculum to be used.

In some embodiments, additional supply lumens 206 are envisioned, for example such as described in co-owned US Pat. Appln. No. 2017/0224379, which is hereby incorporated by reference in its entirety for all purposes.

In some embodiments, lavage fluid is collected in a non-embryotoxic glass or plastic collection bottle at volumes expected to be in a range of 5 and 250 cc's. The lavage fluid may then be diluted in additional physiologic transport media (for example—Hepes based HTF with up to about 20% protein), and the resulting mixture containing embryos is sealed in the collection transport trap with a tightly fitting seal, e.g., glass non-perforated stopper. Further details of the collection trap and transportation processes can be found in US Pat. Appln. Pub. No. 2017/0224379.

In some embodiments, lavage fluid may be collected directly into a cell-culture system capable of maintaining blastocyst viability during shipment to the central embryological laboratory. Such a system maintains blastocyst-safe O2/CO2 levels without the requirement of an incubator and eliminate the need to dilute lavage fluid with physiologic transport media thus streamlining the recovery and shipment of pre-implantation embryo captured through uterine lavage. Once the lavage is complete, embryos may be recovered in a central embryological laboratory, for example.

Turning to FIGS. 7-9, the internal manifold and interaction between return lumen 205 and supply lumen 206 is described. As shown in the figures, housing 202 may include proximal end 214, from which extension tubing 212 extends. Housing 202 is shown to include a handle 213. FIG. 7 shows a simplified cross-sectional view of a portion of housing 202, in order to show interaction between supply lumen 206, return lumen 205, and how supply lumen 206 may exit through a wall of return lumen 205. As shown in the enlarged view of FIG. 8, external to return lumen 205, a seal housing 216 may be disposed on an outer surface of return lumen 205 and enclose a portion of return lumen 205 and fluid supply lumen 206 at a point at which fluid supply lumen 206 exits return lumen 205. As shown, housing 202 encloses a portion of the supply and return lumens 206/205, such that supply lumen 206 exits return lumen 205 along a first housing axis. Return lumen 205 may exit housing 202 to along a second housing axis offset from the first housing axis. Return lumen 205 may include in some embodiments, a radius of curvature 215 between the first housing axis and second housing axis, such that recovered blastocysts 88 freely flow through the return lumen 205. In some embodiments, the fluid supply lumen 206 exits the return lumen 205 in a portion of the radius of curvature 215. Radius of curvature 215 is between about 25 mm and 100 mm, in some embodiments.

As shown, return lumen 205 may be disposed coaxially and surrounding supply lumen 206 along a first axis. Supply lumen 206 may exit the return lumen 205 along the first axis. Return lumen 205 in turn may exit housing 202 along a second axis corresponding to a catheter device handle axis that is offset from the first axis. Radius of curvature 215 may be disposed between the first axis and second axis. In some embodiments, seal housing 216 is disposed on an exterior of the radius of curvature 215 of the return lumen 205. Uterine lavage catheter device 200 may further include a first seal element 600 disposed within a cavity of seal housing 216. First seal element 600 may be disposed around supply lumen 206, and may include an o-ring. In some embodiments, additional seal element 602 may be included, and may also be a o-ring. In this way, supply lumen 206 translates through return lumen 205, without either lumen leaking through handle or housing 202.

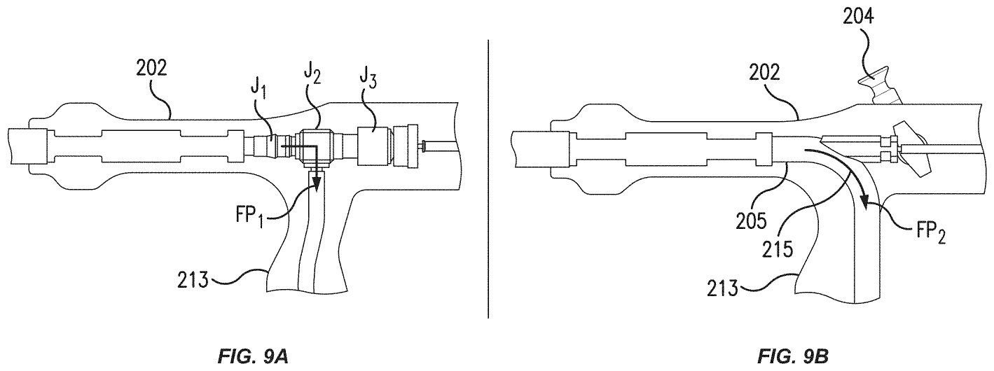

This design is optimized for smooth fluid flow, and eliminates unnecessary fittings that would otherwise lead to stagnation zones (e.g., areas within the device with poor or no fluid flow where fluid, cells, blastocysts 88, etc. may become trapped). The difference between prior manifold design in the instant manifold design can be seen in FIG. 9, for example. On the left-hand side of FIG. 9, flow path FP1 is shown, denoting recovered lavage fluid and blastocyst 88 flow path through return lumen 205. As shown, the left hand side includes three joints (J1, J2, and J3), each being potentially susceptible to stagnation zones. Fluid transfer consisting of flow restrictions and angular fittings are inherently bad for flow and create many stagnation sites. In contrast, on the right-hand side flow path FP2 is shown. Flow path FP2 is a smooth, unobstructed flow path, that eliminates stagnation zones within housing 202.

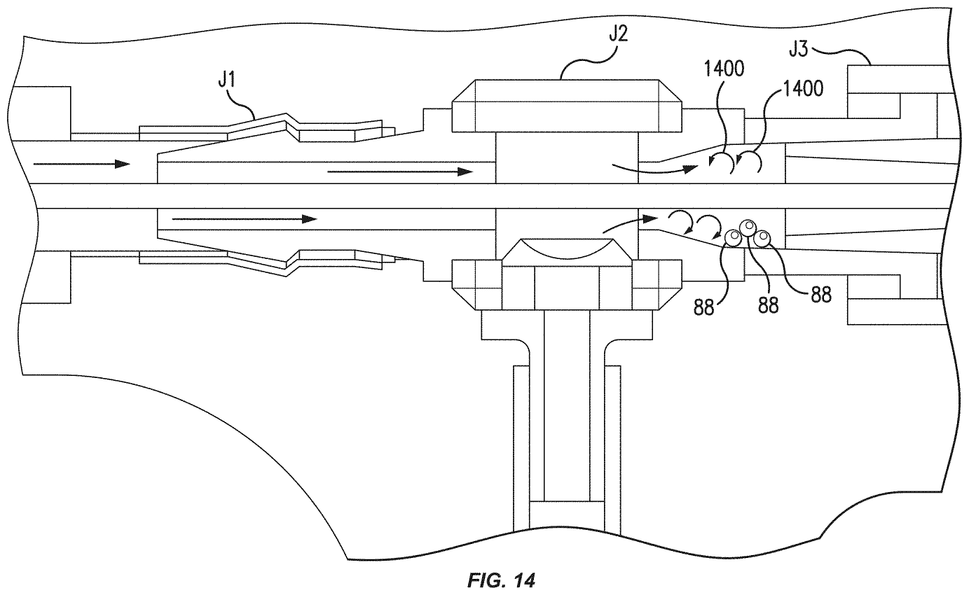

This offers advantages over a conventional manifold, such as one that is a collection of fittings put together to direct fluid flow within a catheter. Such a design, that leverages the smooth transition of radius of curvature 215 and ported manifold feature of intersecting coaxial lumens is desirable over catheters containing manifolds because it reduces or eliminates presence of stagnation sites (which may centrifugally displace the blastocysts from the fluid flow) (FIG. 9B and FIG. 14). Additionally such a design shown on the right-hand side of FIG. 9 (9A) reduces or eliminates restrictions of fluid flow by avoiding features (e.g., corners, edges, right angles) which inhibit fluid flow. The return lumen 205 within a portion of catheter device 200 may be configured as one continuous tube, defining no zones for embryos to get lost, and having a smooth uninterrupted channel. FIG. 14 shows stagnation flow lines 1400 and portions where cells, e.g., blastocysts 88 may be parked in other manifolds (see also FIG. 9B).

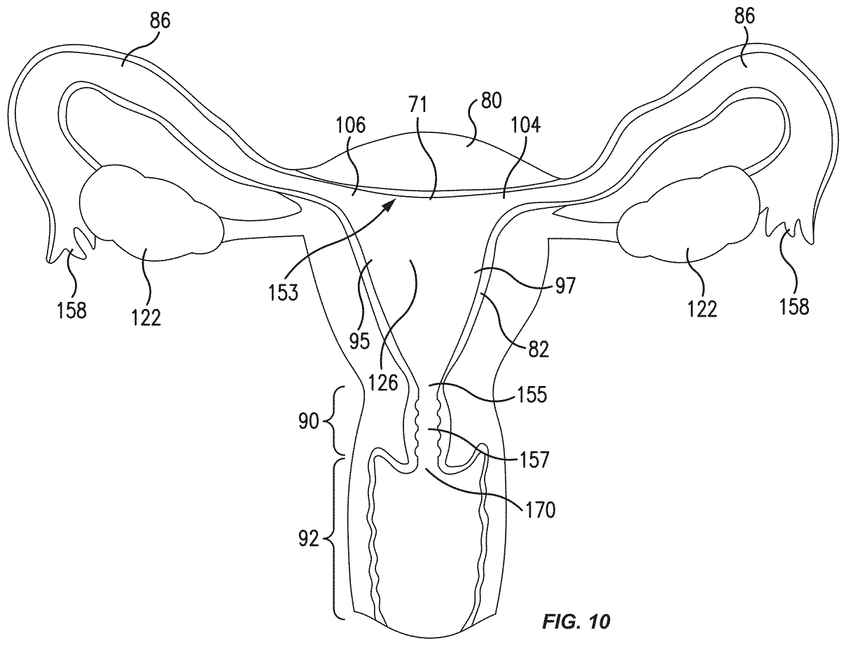

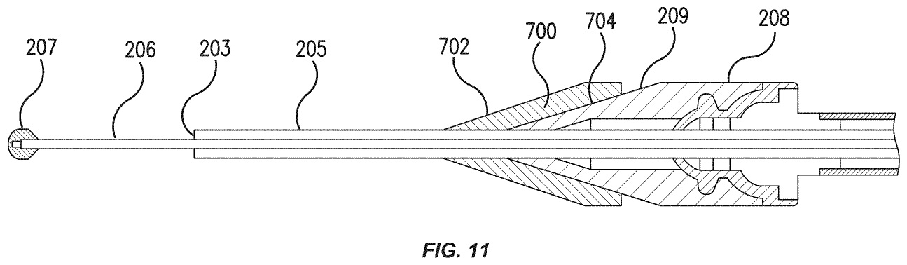

Turning to FIG. 11, extension element 700 is shown and described. In some embodiments, uterine lavage catheter device 200 includes extension element 700 extending from, and in contact with, seal element 208. Extension element 700 includes distal surface 702 that comprises the sealing surface, wherein the extension element is configured to shorten first distance, that is, the distance between the sealing surface and inlet of return lumen 205. In some embodiments, distal surface 702 is conical. As shown, extension element 700 may further include surface 704, which is defined, for example, by a portion of a cavity towards the proximal side of extension element 700. Surface 704 may engage and/or contact surface 209 of sealing element 208. In some embodiments, surface 704 need not include a cavity, and instead may be defined by surface towards the proximal end of extension element 700.

In some embodiments, extension element 700 may be formed from a silicone rubber, for example, or any suitable elastomeric material such as urethane, PEBAX, C-Flex, latex, urethane, santoprene, etc. Extension element 700 is structurally configured to reduce the effective length on the catheter tip such that the central collection opening of the inlet of return lumen 205 is aligned with the internal cervical ostium of the patient's anatomy, e.g. for a patient having a smaller distance to internal cervical ostium. In some embodiments, the element 700 may be spring-loaded, for example, such that a certain degree of flexibility may be afforded e.g. by approximately 1 cm. In some embodiments distal surface of extension element 700 may be conical.

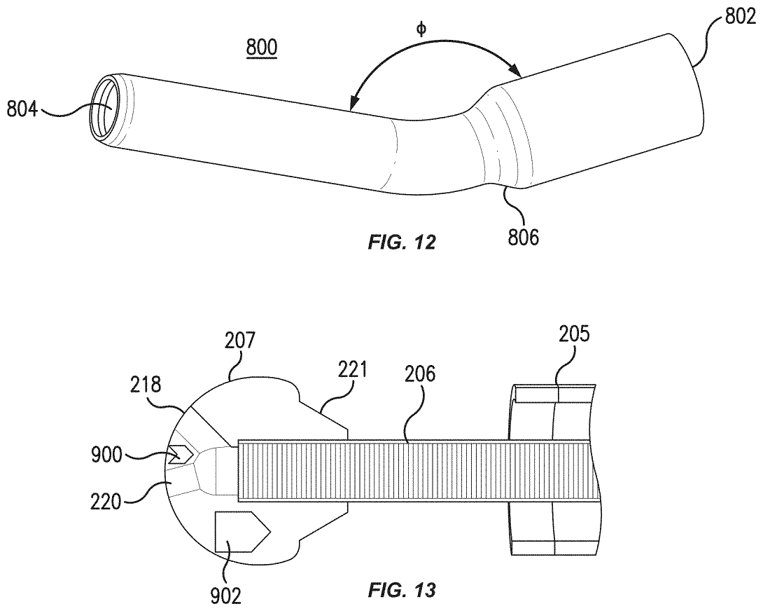

Turning to FIG. 12, tip direction fitting 800 is shown. In some embodiments tip direction fitting 800 is configured as an accessory to catheter device 200 that does not include a steerable feature. Tip direction fitting 800 is configured to be fitted onto catheter device 200, in order to pre-bend catheter device 200. In this way, tip direction fitting 800 provides fixed steerability, which may be used in combination with catheter device 200 rotation for example.

In some embodiments, the angle Φ of the fitting can be varied, or fixed at the manufacturer. In some embodiments, the rotation of tip direction fitting 800 may be varied, e.g., when installing tip direction fitting 800 onto catheter device 200, however angle Φ may be fixed. In some embodiments, angle Φ may be varied, or altered by physician, e.g. by bending a transition portion 806 of tip direction fitting 800. In some embodiments, tip direction fitting 800 includes fluid ports 802 and 804, which may be configured as inlet and/or outlets, respectively. Further angles obtained by the speculum may be accentuated. In some embodiments, angle Φ of the distal portion of the return lumen 206 may be preset and may vary from, for example between about 0 degrees and about 45 degrees from a longitudinal axis of catheter device 200, and is customized to individual women in order to accommodate the different anatomical variations of the uterine flexion. In some embodiments, a plurality of tip directing fittings may be supplied to the user with a variety of angles Φ.

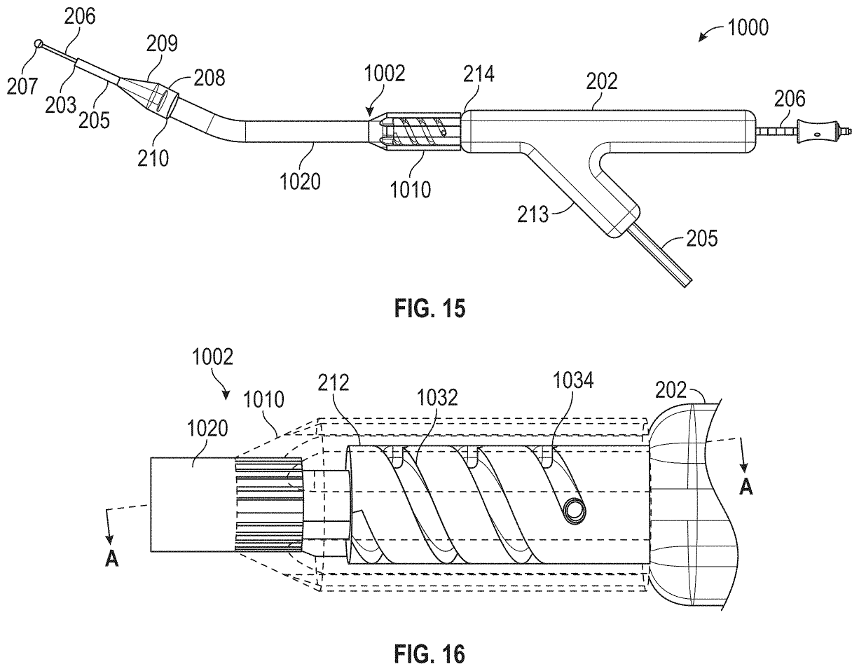

FIG. 15 shows a schematic side view of uterine lavage catheter device 1000 according to an embodiment of the present disclosure. Catheter device 1000 shown in FIG. 15 may include the same or similar features of other embodiments described herein, including seal element 208, supply lumen 206, nozzle 207, and return lumen 205. However, in the illustrated embodiment, catheter device 1000 further comprises an extension assembly 1002 operatively connected to seal element 208 and configured to move seal element 208 in a longitudinal direction along catheter device 200 to adjust the position of the return lumen 205 with respect to internal cervical ostium 155.

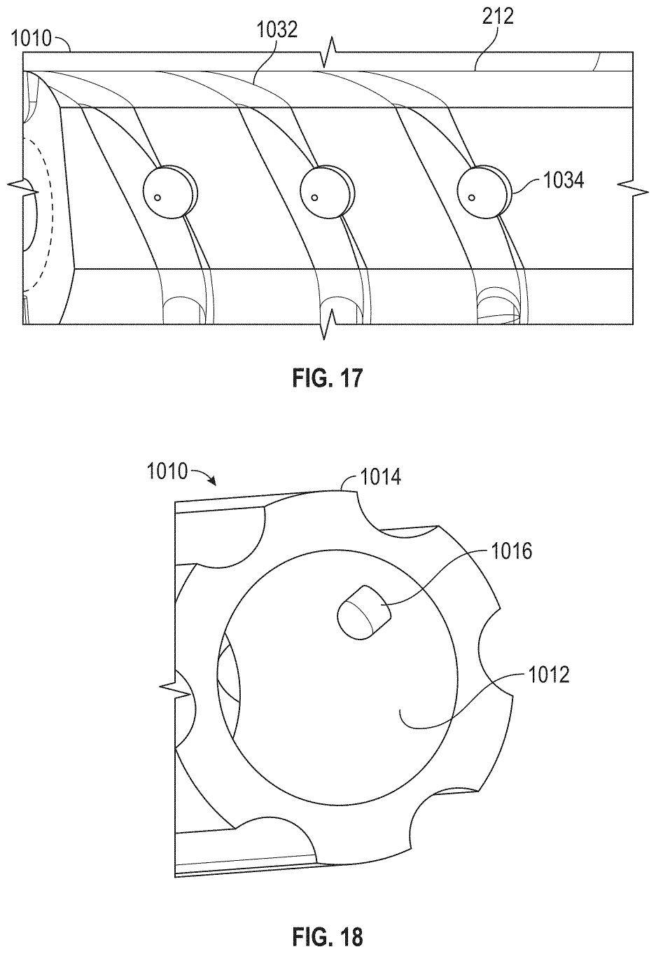

Referring to FIGS. 15-18, extension assembly 1000 includes the extension tubing 212 (FIG. 16) housing a portion of return lumen 205 and supply lumen 206, an actuator 1010 disposed adjacent to proximal end 214 of housing 202, and a flexible outer tube 1020 comprising a first end coupled to actuator 1010 and a second end coupled to seal element 208. In some embodiments, flexible outer tube 1020 is disposed coaxially with extension tubing 212 and configured to move along extension tubing 212 to move seal element 208 in a longitudinal direction. In some embodiments, actuator 1010 is a tubular-shaped knob disposed coaxially with extension tubing 212 and configured to trigger movement of flexible tube 1020 by translating rotary movement about extension tubing 212 to linear movement of flexible tube 1020 in a longitudinal direction along catheter device 1000.

In some other embodiments, extension assembly 1002 comprises a helical-shaped spring disposed between actuator 1010 and extension tubing 212, where the spring is configured to maintain tension against the seal element 208 by biasing the actuator 1010 against the flexible outer tube 1020. The spring may function as a passive control to maintain tension on the seal element, or may include the pin features as described below with reference to FIG. 18.

In some embodiments, as shown in FIG. 18, actuator 1010 comprises a corrugated exterior surface 1014 for promoting grip by a user and a locking pin 1016 projecting in axial direction from interior surface 1012. In some embodiments, pin 1016 may be flexibly coupled to actuator 1010, e.g., via material properties, spring engagement, or the like. In some embodiments, extension tubing 212 comprises a plurality of notches 1034 formed in the outer surface of extension tubing 212 and spatially separated in a longitudinal direction. Each notch 1034 opens into recess 1032 and is configured to receive locking pin 1016 to lock actuator 1010. In some embodiments, spiral-shaped recess 1032 and plurality of notches 1034 may be formed in a sleeve received over the extension tubing 212 or a collar protruding from the extending tubing 212 in an axial direction. In some embodiments, pin 1016 may engage spiral-shaped recess 1032 and be directed into one of notches 1034.

In some embodiments, as the actuator 1010 is rotated about extension tubing 212, locking pin 1016 is configured to move out of one of notches 1034 and slide along spiral-shaped recess 1032 until being received in the next notch 1034. Accordingly, the combination of locking pin 1016 of actuator 1010 and plurality of notches 1034 of extension tubing 212 limit movement of actuator 1010 so that the seal element 208 may move in incremental positions along the catheter device 1000.

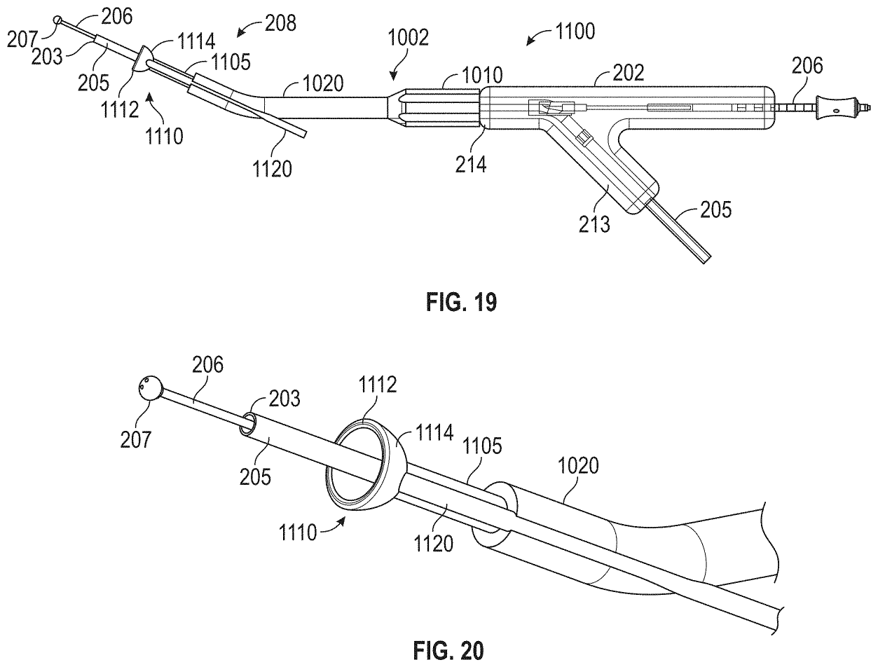

FIG. 19 shows a schematic side view of uterine lavage catheter device 1100 according to an embodiment of the present disclosure. Catheter device 1100 shown in FIG. 19 may include the same or similar features of other embodiments described herein, including seal element 208, supply lumen 206, nozzle 207, return lumen 205, and extension assembly 1002. However, in the illustrated embodiment, seal element 208 comprises a sleeve 1105 disposed coaxially with return lumen 205 and extension tubing 212, a cup seal 1110 disposed at a distal end of sleeve 1105 and spatially separated from inlet 203 of return 205, and a vacuum lumen 1120 extending into the cup seal 1110 so that the cup seal 1110 is configured to be pressure fitted against external cervical ostium 170.

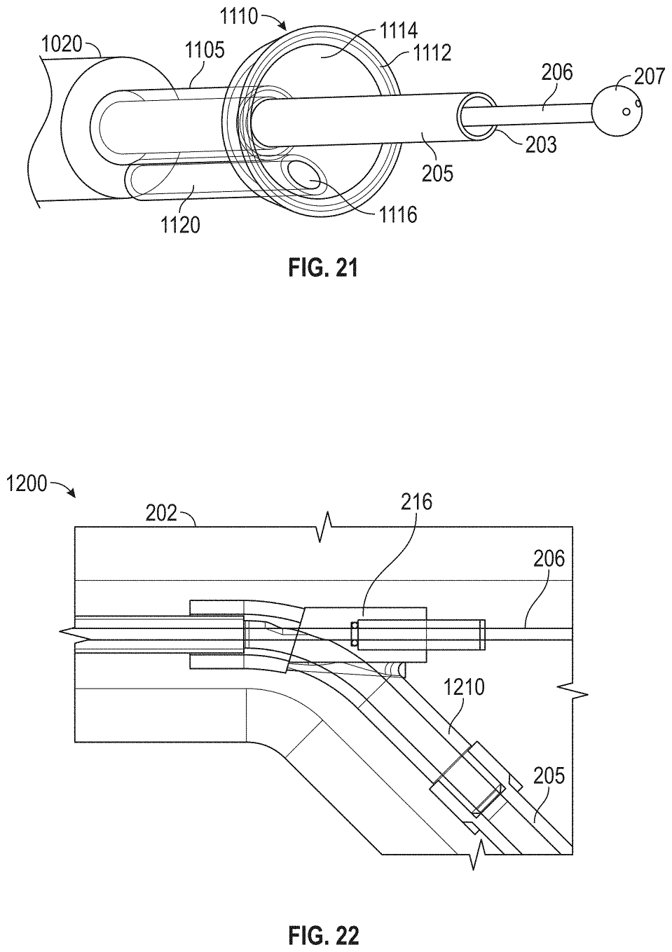

In some embodiments, as shown in FIGS. 19-21, sleeve 1105 comprises a proximal end coupled to second end of flexible tube 1020. In some embodiments, cup seal 1110 includes a circular-shaped rim 1112 and a hemispherical-shaped wall 1114 extending from rim 1112 to the distal end of sleeve 1105, such that seal 1110 defines a cavity enclosed by wall 1114 and opening through rim 1112. In some embodiments, rim 1112 includes a diameter in a range between 25 mm and 45 mm. In some embodiments, cup seal 1110 includes a vacuum port 1116 disposed along wall 1114 and opening into cavity of cup seal 1110. Vacuum port 1116 is configured to be connected to vacuum lumen 1120 for exhausting air out of the cavity. In various embodiments, cup seal 1110 is comprised of a low durometer elastomeric material. In some embodiments, vacuum lumen 1120 is connected to a vacuum source (not shown) configured to generate a pressure differential between an inlet and an outlet of vacuum lumen 1120, such that the vacuum lumen 1120 exhausts air out of the cavity of cup seal 1110, thereby pressurizing the cavity of the cup seal 1110. While the illustrated embodiment shown in FIGS. 19-21 shows the cup seal 1110 comprising a hemispherical shape, in other embodiments (not shown), cup seal 1110 may comprise other suitable shapes for defining a cavity and providing a seal surface.

In operation, seal element 208 may seal exterior cervical surface 170 by engaging rim 1112 of cup seal 1110 against a cervix surface surrounding the exterior cervical ostium 170 and exhausting air out of the cavity of cup seal 1110 through vacuum port 1116 and vacuum lumen 1120. Once air is exhausted out of the cavity of cup seal 1110, rim 1112 of cup seal 1110 is pressure fitted against the external cervical surface, such that cup seal 1110 hermetically seals exterior cervical surface 170. In various embodiments, the position of seal cup 1110 may be adjusted by using actuator 1010 of extension assembly 1002 to move seal cup 1110 in a longitudinal direction along catheter device 1100.

FIG. 22 illustrates a schematic cross-sectional view of catheter device 1200 according to an embodiment of the present disclosure. Catheter device 1200 shown in FIG. 22 may include the same or similar features of other embodiments described herein, including seal element 208, supply lumen 206, nozzle 207, return lumen 205, and extension assembly 1002. However, in the illustrated embodiment, catheter device 1200 includes a second seal housing 1210 disposed in handle 213 of housing 202 and received around return lumen 205. As shown in FIG. 22, second seal housing 1210 is engaged against sealing housing 216 at about transition point 215 to ensure that the return lumen 205 is hermetically sealed. In some embodiments, second seal housing 1210 is comprised of an elastomer material.

In some embodiments, catheter device 200 may include a tenaculum stabilizer, which may include a fixture on catheter device 200 configured to hold a tenaculum (for example, one clamped to a patient's cervix) in order to hold and stabilize catheter device 200 and maintain the seal against cervix during the lavage procedure.

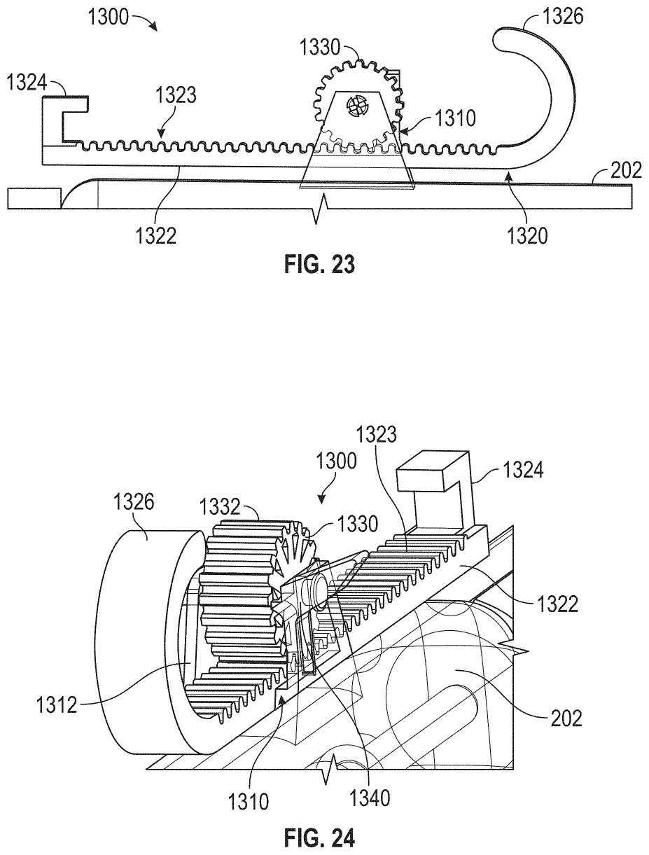

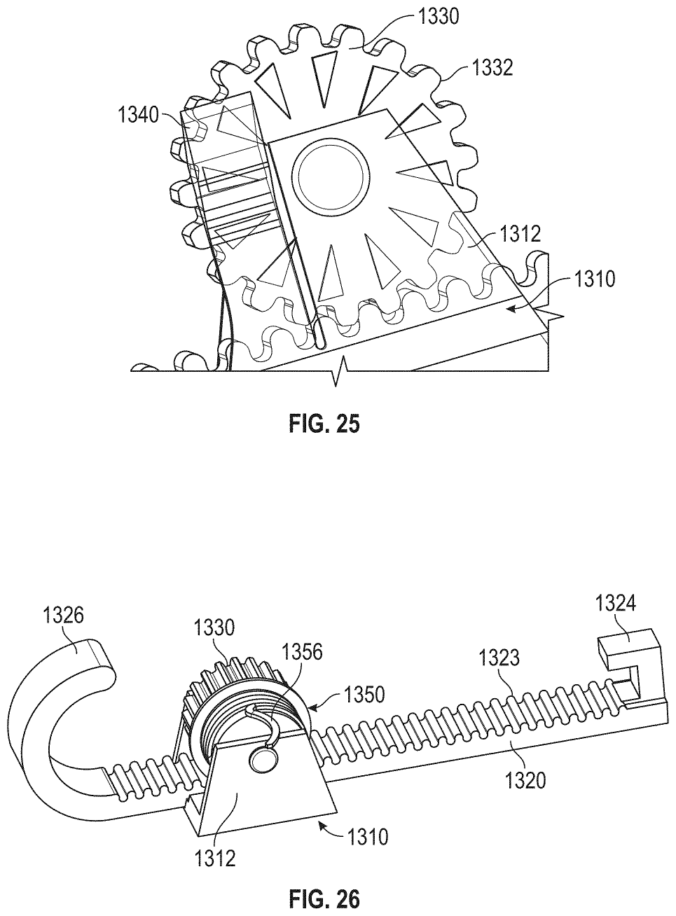

Referring to FIGS. 23-27, catheter device 200 includes a ratchet tenaculum stabilizer 1300 disposed along a top of housing 202 and configured to hold and restrict movement of a tenaculum in one direction. In some embodiments, ratchet tenaculum stabilizer 1300 includes a yoke 1310 coupled to the top of housing 202 and comprising a pair of arms 1312 projecting away from housing 202. In some embodiments, ratchet tenaculum stabilizer 1300 includes a rack 1320 disposed between pair of arms 1312 of yoke 1310 and extending parallel with respect to the housing 202. In the illustrated embodiment, rack 1320 comprises a track 1322 defining a plurality of teeth 1323, a seat 1324 disposed at a first end of track 1322, and a curved handle 1326 disposed at a second end of track 1322. In some embodiments, ratchet tenaculum stabilizer 1300 includes a gear wheel 1330 suspended between pair of arms 1312 of yoke 1310. In the illustrated embodiment, gear wheel 1330 comprises a plurality of teeth 1332 and configured to rotate such that plurality of teeth 1332 engage plurality of teeth 1323 of rack 1320, thereby moving rack 1320 in a longitudinal direction.

In some embodiments, as shown in FIG. 24, ratchet tenaculum stabilizer 1300 comprises a pawl 1340 configured to engage plurality of teeth 1332 of gear wheel 1330 and limit rotary of gear wheel 1330 in one direction (e.g., either clockwise or counterclockwise). In the illustrated embodiment, pawl 1340 is configured to permit rotation of gear wheel 1330 in response to the application of force against handle 1326. In some embodiments, pawl 1340 is configured to pivot toward or away from gear wheel 1330

In operation, ratchet tenaculum stabilizer 1300 is configured to hold a tenaculum against seat 1324 and handle 1326. Ratchet tenaculum stabilizer 1300 allows a user to adjust grasp of tenaculum by pulling handle 1326 in a linear direction away from seat 1324. Ratchet tenaculum stabilizer 1300 allows a user to release tenaculum by pivoting pawl 1340 away from gear wheel 1330 such that pawl 1340 becomes disengaged with plurality of teeth 1332.

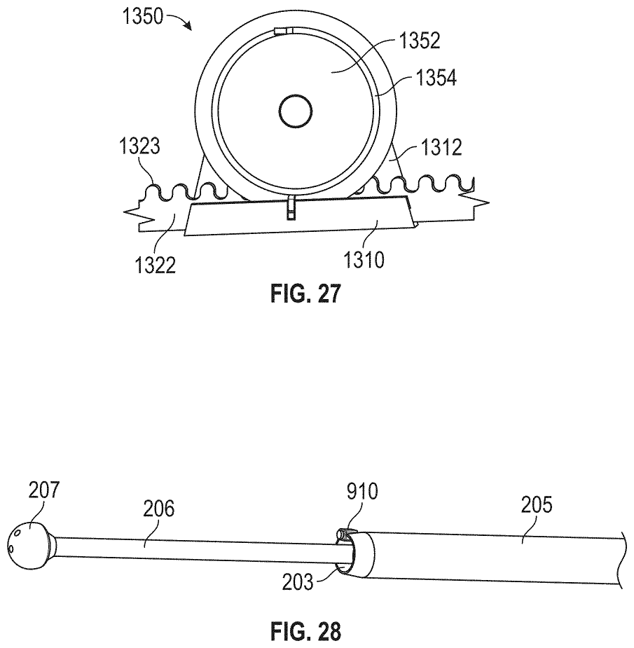

In some embodiments, as shown in FIGS. 26 and 27, pawl 1340 of ratchet tenaculum stabilizer 1300 is replaced with a spring clutch assembly 1350 disposed between pair of arms 1312 of yoke 1310 and configured to restrict rotary movement of gear wheel 1330. In some embodiments, spring clutch assembly 1350 comprises a drum 1352 axially aligned with gear wheel 1330 and operatively connected to gear wheel 1330. In some embodiments, spring clutch assembly 1350 comprises a torsion spring 1354 received around drum 1352. In some embodiments, spring clutch assembly 1350 comprises a spring brake 1356 connected to torsion spring 1354 and extending transverse with respect to drum 1352. In various embodiments, as rack 1320 is pulled in a direction away from seat 1324, torsion spring 1354 is configured to contract and lock rotation of drum 1352, thereby stopping rotary movement of wheel 1330 so that rack 1320 cannot be pulled further back. In various embodiments, as spring brake 1356 is released, torsion spring 1356 is configured to expand and permit rotation of drum 1352, so that rack 1320 may be pulled further back to release tenaculum.

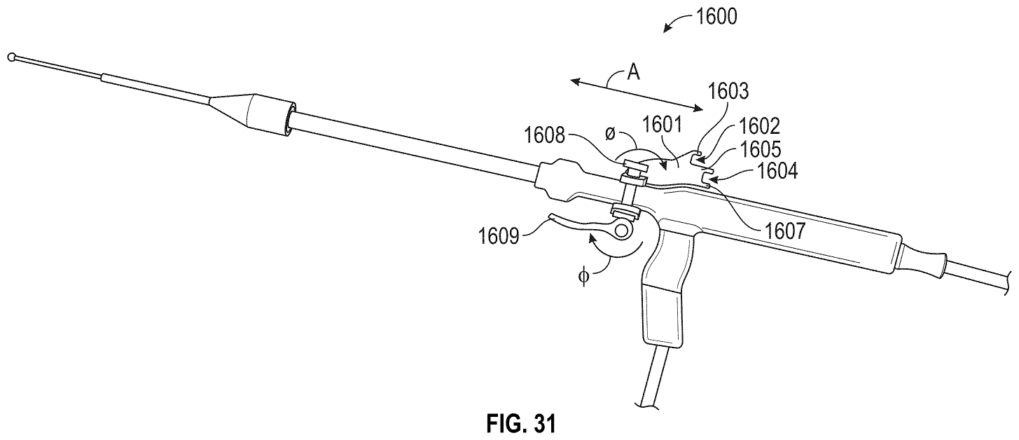

In FIG. 31, a schematic side view of uterine lavage catheter device 1600 is shown according to an embodiment of the present disclosure. Catheter device 1600 shown in FIG. 31 may include the same or similar features of other embodiments described herein, including seal element 208, supply lumen 206, nozzle 207, return lumen 205, and extension assembly 1002. Additionally, catheter device 1600 includes tenaculum stabilizer 1601, which may be configured as an independent accessory. Tenaculum stabilizer 1601 may be attached to catheter device 1600, or other embodiments of the catheter device described herein. As shown in the figure, tenaculum stabilizer 1601 is attached to the catheter device using knob/screw 1608, with clamp 1609 being able to actuate a cam action and create a clamping force to lock tenaculum stabilizer 1601 in place. Knob/screw 1608 is rotatable in the θ direction, while clamp 1609 being rotatable about a pivot in the φ direction.

Tenaculum stabilizer 1601 includes one or more saddles 1602/1604, formed between steps 1603, 1605, 1607. Saddles 1602/1604 form bases for distal and proximal translation, correlating to force pulling on the cervix when the device is fixed and the tenaculum web is positioned within the saddles.

Turning to FIG. 13, an imaging system for catheter device 200 is shown. In some embodiments, catheter device 200 includes a light source 900. Light source 900 may be, for example an LED light source. In some embodiments light source 900 may illuminate a portion of the uterus, for example during insertion of catheter device 200 or during a lavage cycle. Illumination using light source 900 may be controlled remotely, for example by the physician performing the lavage procedure, or separately by an additional person. In some embodiments, controls for light source 900 may be integrated into housing 202, or handle 213 (see FIGS. 4, 9A, 9B). In some embodiments, catheter device 200 may include an imaging sensor, such as camera 902. Camera 902 may be configured to image a portion of the uterus, for example the portion that is illuminated by light source 900. Camera 902 may include a one or both of a CCD and CMOS sensor, for example. One or both of camera 902 and light source 900 may be integrated into one of the lumens of catheter device 200. In this way, they may be configured to visualize the anatomy during insertion of the catheter and the lavage cycle. In some embodiments, one or both of camera 902 and light source 900 may include an additional light source or camera. In some embodiments, any associated wiring, or electronics, or portion thereof, may be integrated within one or more of the lumens of catheter device 200. The use of a camera is advantageous, for example when a physician is unable to obtain sufficient imaging from ultrasound, prior to or during a lavage procedure.

Referring to FIG. 28, the imaging system for catheter device 200 may further include a camera 910 and a light source integrated with return lumen 205, where the camera 910 and light source are disposed proximate to inlet 203. In various embodiments, camera 910 is configured to image a portion of the uterus, such as uterine cavity and any oocytes or embryos present in the uterus.

Some embodiments are directed to a method of recovering oocytes or blastocysts from a human uterus. The method may include inserting a catheter trans-vaginally into a uterus, sealing the exterior cervical ostium with a sealing surface of a seal element, lavaging the uterine walls with lavage fluid with a nozzle, the nozzle is in fluid communication with and coupled to a supply lumen of the catheter. The method may include placing an inlet of a return lumen at the interior cervical ostium by setting a distance between the sealing surface and the inlet of the return lumen, the return lumen positioned coaxially with the supply lumen, and recovering the lavage fluid and oocytes or blastocysts from the uterus with a return lumen disposed coaxially with a supply lumen that supplies the fluid to the nozzle. In some embodiments, the method may include translating the nozzle along the longitudinal axis of the catheter between about 1 mm and 5 mm. In some embodiments, the method may include rotating the nozzle about the longitudinal axis of the catheter such that a first and second fluid outlet are sprayed in a concentric circular pattern. In some embodiments, the method may include bending the return lumen off-axis from the longitudinal axis of the catheter between about −60 degrees and about +60 degrees. In some embodiments, the method may include flowing the lavage fluid through the return lumen through a radius of curvature that intersects a point through which the supply lumen exits the return lumen. In some embodiments, the method may include illuminating a portion of the uterus with a light source coupled to the nozzle, and imaging, via a camera coupled to the nozzle, a portion of the uterus that is illuminated.

Some embodiments are directed to a method of recovering oocytes or blastocysts from a human uterus. The method may include inserting a catheter trans-vaginally into a uterus, sealing the exterior cervical ostium with a sealing surface of a seal element, and lavaging the uterine walls with lavage fluid with a nozzle at a first fluid pressure, wherein the lavaging comprises pulsing lavage fluid between the first fluid pressure and a second fluid pressure different than the first fluid pressure. The method may include placing an inlet of a return lumen at the interior cervical ostium, and recovering the lavage fluid and blastocysts from the uterus with the return lumen, wherein the first fluid pressure is between about 25 mmHg and about 75 mmHg. In other embodiments, the first fluid pressure may range between about 20 PSI and 80 PSI. In some embodiments, the first fluid pressure may be about 40 PSI. In some embodiments, the first fluid pressure may be about 50 PSI.

On arrival in the embryology laboratory, the transported lavage fluid is passed from the transport vial through a filter to remove additional cells and debris and placed into a large flat petri dish. There, it may be scanned by an embryologist using a standard binocular microscope. The blastocysts 88 are recovered by the embryologist using embryological glass pipettes and transferred individually into smaller individual embryological culture (e.g., in Petri dishes) containing standard embryo tissue culture fluid buffered for stability, e.g. Gardner's G-2.2 media). Blastocysts may be biopsied for potential diagnostic tests and/or subsequently cryopreserved for future use of the patients. Due to the inherent variability in the reproductive process, some non-blastocysts (e.g., earlier stage embryos, unfertilized eggs) may also be recovered by the lavage system. Should non-blastocyst embryos be recovered, they may be placed in embryological culture for the time necessary to develop to blastocyst where it can be biopsied and frozen. Some embryos may fail to make it to blastocyst and are normally discarded or donated to research.

Utilizing a micromanipulation apparatus, individual blastocysts 88 may be processed for certain diagnostics, such as molecular genetic diagnosis or sex determination. Further details of such exemplary diagnostics can be found in US Pat. Appln. Pub. No. 2017/0224379. For example, the following methods may be used for the evaluation of chromosomal structures: polymerase chain reaction, whole genome hybridization, microarray gene chips, exome sequencing, or analysis of the entire human genome. In some embodiments, a geneticist evaluates the molecular analysis in combination with information about specific clinical factors of the case. A decision is then made that leads to (a) replacing the embryo in the mother, as unaffected by the disease in question, (b) recommending an intervention such as gene therapy or transplantation of donated stem cells, or (c) recommending that the embryo not be transferred and that another embryo which is unaffected be transferred, e.g., at a later time.

PGT-M allows for identification of embryos that are carriers of genetic disorders or of desired genetic traits. PGT-M facilitates selection of the unaffected or carrier embryos for transfer to (replacement in) the uterus. Embryos afflicted with the genetic disease in question may not be replaced in the uterus and may be discarded per the intended parent's wishes. PGT-A allows identification of embryonic sex. Embryonic sex selection may be used for prevention of sex-linked genetic diseases. Sex selection may also be used for culture, social indications, or family balancing by gender/sex or any combination of the above PGT-SR allows identification of chromosomal restructuring.

Additionally, embryonic gene and stem cell therapy have been achieved in experimental and domestic animals, in human adults and children. Gene and stem cell therapy targeted at the preimplantation embryo is especially promising because it repairs cells with abnormal genetics before differentiation of the cells, by adding to, replacing, or manipulating (or a combination of them) a dysfunctional sequence of DNA. Also, human gene therapy may readily be delivered by blastocoel injection because blastocoel gel comes into direct contact with virtually all cells. Human gene therapy at the blastocyst stage though not yet achieved, is foreseeable in the future, particularly with recent adult human successes with treatment of genetic diseases by gene therapy, e.g. Hemophilia B.

One technique potentially useful at the blastocyst stage is to remove a few stem cells from the inner cell mass, transfect the cells directly using a retroviral vector or by actual micro insertion of the construct into the isolated stem cell. Once the correction is incorporated into the genome of the stem cell, it can be reintroduced back to the inner cell mass where it would be incorporated into the growing embryo. Since the transected stem cells are totipotential, the corrected genetics can be incorporated into any organ including germ cells then transmitted to future generations.

Embryos suitable for replacement in the uterus, either because they are genetically unaffected or have been successfully treated, are cryopreserved for transfer either in the following spontaneous menstrual cycles or at a more remote future date.

Following cryopreservation, embryos suitable for replacement are thawed and transferred back into the uterine cavity. To do this, the embryo is suspended in tissue culture fluid. The fluid is loaded into an embryo transfer catheter. This catheter can be any one of many commercially available devices widely used for embryo transfer in fertility clinics for this purpose. The embryo transfer catheter is passed through the cervix by the same technique commonly used in fertility clinics for in vitro fertilization. Further details of such a process can be found in US Pat. Appln. Pub. No. 2017/0224379. The embryo ultimately implants in the uterine lining 82 (endometrium) (FIG. 10), accesses the maternal blood supply, and then develops for a normal gestation period resulting in the birth of a newborn free of the genetic disorder under treatment.

We have described examples of the procedure in a series of steps performed on a single patient. In making this procedure available to a very large number of patients all over the world (including in large and small communities, and in rural and urban areas), techniques can be applied to reduce the cost, improve the safety, and enhance the efficiency and performance of the procedure, among other things. One or more appropriate business models can be used to provide these advantages to patients while offering revenue and profit opportunities for manufacturers and distributers of the devices used in the procedure, providers of the services that are part of or associated with the procedure (including PGT-A, PGT-M, PGT-SR, PGD, PGS, genetic disease prevention, embryonic gene therapy, and stem cell transplantation), medical professionals, and other parties. The business model can include a variety of transactional features including sale, rental, and licensing of devices and equipment, fees for services, licensing of services, and others. Further details of such models can be found in US Pat. Appln. Pub. 2017/0224379.

In some implementations, one or more components of the catheter device 200, such as return lumen 205, supply lumen 207, and nozzle 207, may be one time use disposables. In some embodiments, one or more of the components are configured as kits to be sold. In some embodiments, the kits may also comprise superovulatory drugs and/or GnRH antagonists. Such kits may include but are not limited to a kit for uterine lavage comprising a uterine lavage catheter device configured for insertion into a woman's uterus to remove viable blastocysts; and one or more containers comprising a sufficient dosage amount of GnRH antagonist to cause desynchronization through corpus luteum apoptosis. Such kits may also include other containers with compounds and/or devices, in lieu of the GnRH antagonist, which can also cause desynchronization of the uterus. Further details of such compounds and/or devices can be found in U.S. application Ser. No. 14/943,678.

In one embodiment is provided kit for uterine lavage comprising a uterine lavage catheter device configured for insertion into a woman's uterus to remove viable blastocysts from the uterus; one or more first containers comprising a sufficient dosage amount of FSH appropriate for induction of superovulation; one or more second containers comprising a sufficient dosage amount of a GnRH antagonist to silence the ovaries while causing superovulation; one or more third containers comprising a GnRH antagonist to be administered after superovulation.

Both permanent (reusable) and disposable (one time use) elements and related support services will have commercial application and market potential outside of preimplantation genetics.

Examples of applications of intrauterine lavage and the devices that we have described, outside of the network system could include the following: 1) Embryo donation: Uterine lavage can be used as a nonsurgical method for embryo donation that will compete with IVF. The availability of newer safeguards to protect donors from sexually transmitted viral diseases allow uterine lavage to be used as a simpler and less expensive alternative. 2) Embryo banking: Uterine lavage is a useful technology allowing couples wishing to defer child bearing to cryopreserve and bank their own embryos for the benefit of career ascension, for example. An additional use is deferred use in anticipation of technical advancements in genetic screening and gene therapy for a condition or disease for which there was no effective treatment at the time of the initial blastocyst recovery 3) Oncofertility: Uterine lavage finds application for patients with malignancies who wish to cryopreserve and bank their own embryos prior to cancer therapy. 4) Diagnosis of fertility and pregnancy wastage disorders: Uterine lavage is useful in embryonic diagnosis of various fertility and pregnancy wastage disorders by facilitating recovery and diagnostic manipulation of preimplantation embryos conceived in vivo. 5) Advanced maternal age: Uterine lavage find applications for older patients >35 who are seeking to get pregnant but due to their age have increased difficulties of conceiving naturally. Uterine lavage may help these patients to cryopreserve their embryos so that they can be used for transfer at a later date.

Uterine lavage may help lesbian couples conceive by making possible the recovery of embryos from one partner, and the transfer of said embryo(s) into the other partner. Currently a similar procedure is performed through IVF but a surgical step is needed to retrieve the oocytes, said oocytes are fertilized and the embryo cultured for up to six days; following completion of embryo culture the embryos are transferred into the recipient partner. Uterine lavage simplifies this process by avoiding the surgical procedure and in vitro incubation period. 7) Fertility Preservation. Uterine lavage can help enable fertility preservation through the recovery of oocytes from the uterus. As above, the lavage procedure described herein may be performed within 0 to 36 hours of natural ovulation (or within 0 to 24 hours, 12 to 36 hours, or 24 to 36 hours of natural ovulation) to recover oocytes that are present in the uterus. In various embodiments, the lavage procedure described herein may be performed within 36 to 72 hours after the application of an ovulatory trigger (or within 36 to 60 hours, 48 to 72 hours, or 48 to 60 hours after application of an ovulatory trigger). Oocytes can be frozen for future use of the patient. 8) Single Women Fertility. Uterine lavage can help single women interested in pregnancy conceive. Currently IUI or IVF with a sperm donor are used for single women fertility, uterine lavage can serve as an alternative option for reproduction that allows for selection of embryos that are transferred back into the uterus.

General construction and clinical operation of examples of a device useful for intrauterine lavage are provided. The principles of construction, operation, and use represented by the examples described and shown here can also be implemented in a wide variety of other examples.

In some embodiments, the systems and methods described herein may utilize an operating frame. Further details of the operating frame, and interaction with the systems and methods described herein can be found for example in US Pat. Appln. Pub. No. 2017/0224379. In some embodiments, each of the system components, or two or more of them in combination may be incorporated in a device or parts of a device and a procedure or parts of a procedure without the other features. Each of the three features has significance of its own, as described herein and can be used itself in a wide variety of devices and procedures. In some embodiments, before the lavage, the three components are pre assembled with dimensions and settings that, in some cases, have been predetermined and customized for each woman.

In some embodiments, an operating frame, with the disposable or otherwise uterine lavage components secured to it, is mounted on a rigid stand. The hard stand is a heavy-duty version of a common so-called Mayo table, which is readily available in the commercial marketplace. Such a table can be slightly modified to support the weight of the operating frame. One person manages the lavage, with both hands free to manipulate off and on functions of the external controller and to make adjustments in the collection apparatus. During the procedure, the patient is recumbent lying down and stabilized using soft restraints while the system is in operation. The operating frame stabilizes the systems for cervical and intrauterine insertion of the suction-recovery cannula and its accessories and for steering the fluid supply catheters and their tip(s) before, during, and after lavage-recovery operations.

It is important, during the lavage procedure, that the frame of the instrument be held in a rigid position and orientation relative to the woman's reproductive anatomy. The setting of the position and orientation can be aided by ultrasound and other techniques. Careful positioning and orientation helps to assure that the lumens lie at an effective insertion distance within the woman and is properly seated by the giving surfaces and with a good fluid-tight seal provided. During catheter insertion, because the instrument is held in an essentially fixed position and orientation relative to the woman's reproductive anatomy, the person performing the procedure can safely and effectively deploy and remove the catheter(s).

In some embodiments, return lumen 205 may be a seamless conduit leading to the recovery portion. In some embodiments, additional conduits may be provided as part of return lumen 205. In some embodiments, accessory channels may be imbedded within return lumen 205. Accessory channels may be provided (depending on the implementation) to guide the deployment of supply lumen 206 into the uterus. Other accessory channels may be provided, for example in embodiments that rely on intracervical inflatable collars (for example as discussed in US Pat. No. 2017/0224379)

The sizes and shapes of the catheter devices 200, including nozzle 207, supply lumen 206, and return lumen 207 (which may be disposable items) are selected to fit the patient and achieve effective lavage. These components may be connected to an external controller programmed to both deliver lavage liquid to the uterus and apply vacuum to the lumens, which supply uterine lavage fluid in a pulsed rhythm. A pump is connected to the supply lumen 206. Suction may be applied at the end of lavage and controlled by a pinch valve which vents the collection bottle during lavage. Intrauterine pressure may be controlled, e.g., by the collection bottle height. In some embodiments, a vacuum element alternates suction in pulses cadenced exactly the opposite as pulses used for fluid delivery (that is, when a pulse is applied, the suction is off, and vice versa correct). Suction pulses are managed via means of a pinch valve, for example. Lavage fluid is supplied to the pump from an external reservoir through the intake port of the pump. For example, the pulsing can be done at a preset frequency in the range of one pulse per 0.250 to 4 seconds. The pulse rate is determined empirically to achieve the most effective and efficient flushing of the uterus to produce the maximum embryo yield. The pulse rate is programmed into the controller.

Uterine lavage as described herein is typically performed between 3 and 7 days after insemination. At the optimal time (e.g., day 5), blastocysts 88 are present suspended in uterine fluid in the potential space 126 between the anterior and posterior uterine walls at approximately the geometric center of the uterine cavity. This location is in close proximity to the ultimate site of implantation, which likely would take place within one day or less after the procedure were there blastocysts remaining in the uterus afterward.

Prior to lavage for embryo recovery, and prior to superovulation and insemination, a practice lavage can be performed (approximately one or two months) before the live procedure is scheduled. In the practice lavage, the instruments are custom fitted, the guides, balloons, and other components and devices are attached into place on the operating frame and measurements are taken (with the assistance of imaging technologies) that will enable the anatomy of each patient to be accommodated. Precise imaging of each woman's anatomy utilizes imaging devices, e.g., two-dimensional or three-dimensional ultrasound, magnetic resonance imaging, or other imaging technology. In one example, the length of fluid supply lines including return lumen 205 and supply lumen 206 required to form a complete loop with the confines of the uterine cavity may be determined and recorded. Additional measurements including anatomical distances, angles, and shapes may be taken into account to customize uterine lavage system including components that make up uterine lavage catheter device 200.

On the day of the lavage procedure, prior to the arrival and positioning of the patient, a previously assembled catheter device-operating frame and supporting lavage instrumentation is assembled and set up in the treatment room adjacent to a gynecological examination table. Prior to the patient encounter, instruments are pre-assembled from disposable and reusable elements, and adjusted as determined by the unique characteristics of each woman as previously determined and measured at the time of the trial lavage. Thus disposable components of the right size and configuration are preloaded into their respective positions. The operating frame and associated instruments are firmly secured on a fixed floor mounted hard stand placed at the foot of the gynecological examination table. The pulsing and suction elements are connected. Prior to initiating the procedure, the catheter, and the pulsing/fluid return elements are primed fully with lavage fluid to clear the instrument of any air in the system. This step mitigates any possibility of the exposure to air onto the blastocyst, as well as the possibility of air embolism to the patient. After the priming step, the instrument is ready for the procedure.

In summary, in preparation for the live lavage, the disposable and reusable elements of the instrument are selected based on prior measurements and study of the woman's anatomy and assembled and attached to the pulsing and suction elements, ready for the procedure. In this way, the live lavage is expected to produce the most efficient and effective recovery of embryos possible.

In a live lavage (live in the sense that oocytes or embryos are present), the procedure begins with the patient on her back in a dorsal lithotomy position. After insertion of a sterile vaginal speculum (not shown), the inner walls of the vagina 92 and the cervix 90 are cleansed with sterile tissue culture fluid. The bladder is left distended so that the procedure can be monitored in real-time by abdominal ultrasound. Two hours before the procedure, if needed for a woman with a strictured cervix 90, the endocervical canal 157, as described previously is dilated with a sterile laminaria (“dry seaweed”) expander. To begin the procedure, the endocervical canal is then mechanically dilated, if necessary, to accommodate a #15 to #34 French device.

Lavage-embryo recovery operations are now performed in four steps: 1) Intracervical insertion of the supply lumen and return lumen into the cervix; 2) Sealing the uterus; 3) Intrauterine insertion, steerage and placement of the supply and return lumen and lavage; and 4) Embryo recovery.

The procedure begins when the supply and return lumens are directed through the vagina into through the endocervical canal (FIG. 3). As the device is inserted, sealing surface 209 comes to rest against the exocervix at the external cervical ostium (e.g., 90, 170) and limits the insertion depth of the guide. The system is now in place for deployment of lavage fluid from supply lumen 206.

With the sealing surface 209 pushed firmly against the cervix at the external ostium, sealing off the endocervical canal and prevents any transcervical loss of lavage fluid and embryos.

Once the cervix is sealed, the fluid supply lumen 206 is then guided into the uterine cavity 126 using wheeled steering controls and linkages (which may be mounted on the operating frame and customized to a particular device). The instruments are connected to the controller delivery pump. The pump is energized and a total of, for example, from about 10 ml to about 100 ml (or between about 5 ml to about 200 ml) of pulsating lavage fluid is infused through the system and uterine cavity and recovered over a period of, for example, about 30 seconds to about 5 minutes (or between about 15 seconds to about 10 minutes). The volume of fluid within the uterus is controlled so a volume may not exceed a predetermined threshold, e.g., not to exceed about 10 mL (or between about 5 mL and about 15 mL) at any given time so as to not over pressurize the cavity or cause contractions, which would be painful to the patient and affect the recovery efficiency of the procedure. In some embodiments, the fluid velocity may be about 220 mL/min (or between about 150 mL/min and about 300 mL/min). In this way, catheter device 200 is configured within an optimal range to ensure a high collection of cells, while maintaining safety of embryos/blastocysts 88. In some embodiments, an alarm may be triggered if fluid velocity exceeds a threshold level, for example about 240 mL/min (or between about 150 mL/min and about 300 mL/min). In some embodiments, an alarm may be triggered if fluid velocity drops below a second threshold level, for example about 220 mL/min (or between about 150 mL/min and about 300 mL/min). In some embodiments, fluid velocity may be stopped, for example if fluid velocity drops below a third threshold level, for example about 130 mL/min (or between about 100 mL/min and about 200 mL/min). As described herein, the fluid flow may be pulsed. Pulsatility creates turbulence and inertial effects, which aids in this lodging cells, and blastocysts. In some embodiments, lavage fluid is flowed constantly, and pump settings may be configured to not allow static fluid. In some embodiments, fluid pulses alternate between two distinct flow states. A duty cycle approach for a single speed pump provides inertial flow pulses while maintaining consistent flow.

One or more of the components of the systems may be constructed of medical grade biochemically inert medical grade composite (for example Teflon®, high grade steel, etc.).

In some implementations, the catheter device (and one or more of the other disposable elements) is custom fabricated by the manufacturer for each patient between the time of the test lavage and the time of the live lavage. In some implementations, the catheter device or one or more of the other disposable elements of the instruments are supplied in a number of different sizes and configurations and can be assembled at the clinic without requiring custom manufacturing.

Outside the woman's body, the lavage fluid flow and embryos is directed into a recovery trap attached at the end of line. During the lavage procedure, no embryos are lost via the internal ostia because all flow is directed at the internal cervical ostium. Thus, there is no force or flow strong enough to force the embryos through the internal ostia or into the fallopian tubes where they would be lost. In some environments, flow of fluid is stopped at the end of the procedure and the catheter devices and supportive elements are removed. Other broad terms may be used to refer to the flow of the fluid within the uterus from the delivery of the fluid to the collection of the fluid. For example, the multiple streams emanating from the catheter can form what is called a layer of fluid, or a curtain of fluid or a wash of fluid. We use all of these terms in a broad sense.

In some embodiments, lavage fluid containing embryos is delivered under intermittent suction into the suction. At the end of the lavage procedure, the recovery trap containing the lavage fluid (and relatedly, blastocysts 88) may be marked using electronic identification tags or otherwise identified. The trap then is filled to a full mark with sterile transport media and sealed, e.g. with a glass stopper for transport to the core embryology genetics laboratory facility. The transport flask may be contained inside an insulated transfer block and transported in an insulated carrying case.

Once complete, the instruments are removed and the patient is discharged. The procedure from insertion of the suction cannula to embryo recovery in the trap is expected to take about 15 minutes. The disposable portions of the instrument are discarded as medical waste, and the reusable portions are sterilized for reuse.

We have described a variety of implementations of the devices and techniques that we have introduced above. A wide variety of other implementations, examples, and applications fall within the scope of our concepts.

For example, other approaches to recovering the embryos from the woman's uterus may be possible using other fluid-based and possibly non-fluid-based techniques and combinations of two or more of them. Important goals in whatever techniques are used are to recover essentially all of the embryos that are present in the uterus (which improves the efficiency of the process), to avoid delivering any fluid or other foreign material into the fallopian tubes, to perform the procedure safely and with the least discomfort to the woman, and to perform the procedure in the shortest time and with the least expertise necessary.

Once the embryos are recovered, a wide variety of procedures, diagnoses, screenings and treatments can be applied to them, not limited to genetic diagnosis or sex determination and associated treatment. The embryos could be used for and treated in accordance with any ethical purpose.

When lavage is used to recover the embryos, a wide variety of approaches and parameters can be applied. For example, any fluids or combinations of two or more of them can be used, provided that they are safe and effective and can successfully cause the embryos to be flushed from the uterus. Although we have referred to the fluid as entraining the embryos for removal, other fluidic mechanisms to remove them may be safe and effective, including flushing, spraying, pooling, or any combination of those and others.

When lavage is used to recover oocytes, a wide variety of approaches and parameters can be applied. For example, any acceptable fluid-type or combinations of two or more of them can be used, provided that they are safe and effective and can successfully cause the embryos to be flushed from the uterus. Although we have referred to the fluid as entraining the embryos for removal, other fluidic mechanisms to remove them may be safe and effective, including flushing, spraying, pooling, or any combination of those and others.

We have referred to pulsating the lavage fluid during the procedure, and pulsating and aspiration to remove the fluid from the uterus, possibly in synchronization with the delivery pulses. A wide variety of other regimes may be effective, including no pulsing of the delivery fluid, and profiles of changing delivery pressure and suction that might not be characterized as pulsing. We use the term pulsating broadly to include all of such regimes, for example. Similarly there may or may not be synchronization of the delivery pressure and suction pressure.

We have suggested above that one aspect of achieving a high recovery rate for the embryos is to seal the uterus during the procedure so that essentially none of the lavage fluid leaks out of the woman (possibly with embryos in the fluid). Other techniques that might not be characterized as sealing may be possible to use to achieve a similar high recovery percentage of the fluid and embryos. When sealing is used, the sealing may be done at other locations than at the entry of the cervix into the uterus. In any case, it is considered useful to do the sealing in a manner that is relatively simple, easy to achieve, safe, effective, and can be effected from outside the woman's body by the same person who is performing the other steps of the procedure. Sealing can be achieved in a variety of ways other than providing a sealing surface, extension element, etc., or alone or in combination with an inflatable balloon, including other inflatable or non-inflatable devices or mechanisms. In some examples, it is useful to arrange the sealing device so that it can be inserted in a non-inflated or non-deployed state and then be inflated or deployed.

In many of the examples that we mentioned earlier, the lavage is achieved by multiple streams of fluid aimed toward the center of the uterus. A wide variety of approaches and combinations of them may be possible. In general, a goal is to assure that all parts of the uterus, and especially the central region where the preimplantation embryos tend to be located, are washed by fresh lavage fluid so that every embryo is impacted by the fluid. Then the fluid with the embryos present is collected by any technique that can avoid the loss of embryos.

It is useful as part of the procedure to seat the lavage instrument at a predetermined insertion position relative to the woman's specific anatomy in order for the fluid to be effectively delivered and recovered. We have described examples in which the distance between two elements of the instrument is adjusted according to the distance between the end of the cervix that opens into the vagina and the end of the cervix that opens into the uterus. This technique could be combined or replaced by other techniques for seating the instrument in a position and orientation that permit safe and effective lavage of essentially all of the embryos in the uterus. The seating of the device is useful to assure a good seal against the leakage of fluid, and also to assure that the fluid carrying elements of the device can be deployed easily and effectively and in the best location for lavage.

We have described implementations in which the lavage delivery and recovery elements of the instrument are manipulated and deployed by rotation and extension of those elements relative to a static support. A variety of techniques can be used for deployment in combination with or in substitution for that described approach with the goals of relatively quick and easy deployment, effective lavage, and comfort of the woman, among others.

The examples of lavage instruments that we have described include lavage elements and sealing elements that can be moved, inserted, deployed, manipulated, and later withdrawn relative to a fixed or static portion of the device. In some examples, the lavage and sealing elements ride within a tube that is part of the static device. In some implementations, devices for carrying fluid both for delivery and recovery, and elements that enable manipulation from the proximal end of the tool are located outside the woman during the procedure.

The balloon, if used, could have a non-funnel shape. More than one balloon could be used. The suction drain need not be located in the funnel.

Other implementations are within the scope of the following claims.

For ease of reference, the following key identifies numerals on the figures and related items associated with those numerals.

Top Endometrial Cavity—71

Uterus—80

Endometrial Lining—82

Proximal fallopian Tube—86

Distal fallopian Tube—87

Embryos (Blastocysts)—88

Peritubal Ovarian Interface—89

Cervix—90

Vagina—92

Right Middle Endometrial Cavity—95

Left Middle Endometrial Cavity—97

Tubal Ostium Patient Left—104

Tubal Ostium Patient Right—106

Ovary—122

Oocytes—124

Uterine Cavity—126

Sperm—128

Insemination Catheter—130

Fundus—153

Internal cervical ostium (also referred to as internal os)—155

Endocervical Canal—157

External cervical ostium (also referred to as external os)—170

As used herein in association with a value, “approximately” denotes +/−10% of the value given. As used herein in association with a value, “about” denotes +/−10% of the value given.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not target to be exhaustive or to limit the embodiments to the precise forms disclosed. It will be apparent to one of ordinary skill in the art that many modifications and variations are possible in view of the above teachings.

What is claimed is:

1. A uterine lavage catheter device for recovering blastocysts from a human uterus, comprising:
a seal element configured to provide a sealing surface against the exterior cervical ostium;
a supply lumen extending from the seal element and configured to supply lavage fluid;
a nozzle coupled to the supply lumen; and
a return lumen having an inlet, the return lumen disposed coaxially with the supply lumen,
wherein the inlet of the return lumen is located a first distance from the sealing surface, such that the inlet to the return lumen is configured to be placed at the interior cervical ostium.

2. The catheter device of claim 1, wherein the seal element comprises a conical distal surface that comprises the sealing surface.

3. The catheter device of claim 1, further comprising:
an extension element extending from and in contact with the seal element, the extension element further comprising a distal surface that comprises the sealing surface, the extension element configured to shorten the first distance.

4. The catheter device of claim 3, wherein the distal surface of the extension element is conical.

5. The catheter device of claim 1, the catheter defining a second distance between the distal end of the nozzle and the inlet to the return lumen, wherein the supply lumen is translatable along the shared lumen axis between about 1 cm and 5 cm.

6. The catheter device of claim 1, wherein the supply lumen is rotatable along the shared lumen axis.

7. The catheter device of claim 1, wherein the catheter defines a second distance between the sealing surface and the tip of the nozzle; and a third distance between the sealing surface and the inlet of the suction line such that the difference between the second and third distances is adjustable between about 2 cm and 8 cm.

8. The catheter device of claim 1, wherein the return lumen is the sole suction path for fluid supplied by the fluid supply lumen and blastocysts.

9. The catheter device of claim 1, wherein the return lumen is steerable between about −60 degrees and +60 degrees off-axis.

10. The catheter device of claim 1, wherein the nozzle further comprises:
a first fluid outlet; and
a second fluid outlet angularly offset from the first fluid outlet with respect to the axis of the fluid supply lumen, wherein the angle between the first fluid outlet and the second fluid outlet is between about −60 degrees and +60 degrees.

11. The catheter device of claim 1, wherein the nozzle further comprises:
a first fluid outlet disposed between about 5 degrees and 25 degrees off-axis.

12. The catheter device of claim 11, further comprising:
a second fluid outlet disposed between about 25 and 55 degrees off-axis.

13. The catheter device of claim 1, wherein the nozzle further comprises:
a first fluid outlet; and
a second fluid outlet linearly offset from the first fluid outlet with respect to the axis of the fluid supply lumen.

14. The catheter device of claim 1, wherein the nozzle further comprises:
a first fluid outlet; and
a second fluid outlet, wherein the first and second fluid outlets differ in size or shape.

15. The catheter device of claim 1, further comprising:
a housing enclosing a portion of the fluid supply lumen and the return lumen,
wherein the fluid supply lumen exits the return lumen along a first housing axis,
wherein the return lumen exits the housing along a second housing axis offset from the first housing axis, and
wherein the return lumen includes a radius of curvature between the first housing axis and second housing axis such that recovered blastocysts freely flow through the return lumen.

16. The catheter device of claim 15, wherein the fluid supply lumen exits the return lumen in a portion of the radius of curvature.

17. The catheter device of claim 16, wherein the radius of curvature is between about 25 mm and 100 mm.

18. The catheter device of claim 1, wherein the nozzle further comprises:
a proximal sealing surface configured to seal the inlet of the return lumen when the supply lumen is in a first position.

19. The catheter device of claim 18, wherein the proximal sealing surface extends into the inlet of the return lumen when the supply lumen is in the first position.

20. The catheter device of claim 18, wherein the proximal sealing surface is conical.

21. A method of recovering blastocysts from a human uterus, comprising:
inserting a catheter device trans-vaginally into a uterus;
sealing the exterior cervical ostium with a sealing surface of a seal element;
lavaging the uterine walls with lavage fluid with a nozzle, the nozzle coupled to a supply lumen of the catheter;
placing an inlet of a return lumen at the interior cervical ostium by setting a distance between the sealing surface and the inlet of the return lumen, the return lumen positioned coaxially with the supply lumen; and
recovering the lavage fluid and blastocysts from the uterus with a return lumen disposed coaxially with a supply lumen that supplies the fluid to the nozzle.

22. The method of claim 21, further comprising:
translating the nozzle along the longitudinal axis of the catheter between about 1 mm and 5 mm.

23. The method of claim 21, further comprising:
rotating the nozzle about the longitudinal axis of the catheter such that a first and second fluid outlet are sprayed in a concentric circular pattern.

24. The method of claim 21, wherein the nozzle further comprises:
a first fluid outlet disposed between about 5 degrees and 25 degrees off-axis from the longitudinal axis of the catheter; and
a second fluid outlet disposed between about 25 and 55 degrees off-axis from the longitudinal axis of the catheter.

25. The method of claim 21, further comprising:
bending the return lumen off-axis from the longitudinal axis of the catheter between about −60 degrees and +60 degrees.

26. The method of claim 21, further comprising:
flowing the lavage fluid through the return lumen through a radius of curvature that intersects a point through which the supply lumen exits the return lumen.

27. The method of claim 21, further comprising:
illuminating a portion of the uterus with a light source coupled to the nozzle; and
imaging, via a camera coupled to the nozzle, a portion of the uterus that is illuminated.

28. A uterine lavage catheter device for recovering blastocysts from a human uterus, comprising:
a fluid supply lumen;
a return lumen disposed coaxially and surrounding the fluid supply lumen along a first axis; and
a housing enclosing a portion of the fluid supply lumen and a portion of the return lumen,
wherein the fluid supply lumen exits the return lumen along the first axis,
wherein the return lumen exits the housing along a second axis corresponding to a catheter device handle axis and offset from the first axis, and
wherein the return lumen includes a radius of curvature between the first axis and second axis.

29. The catheter device of claim 28, wherein the radius of curvature is has a curve length between about 25 mm and 100 mm.

30. The catheter device of claim 28, wherein the fluid supply lumen exits the return lumen in a portion of the radius of curvature.

31. The catheter device of claim 28, wherein the radius of curvature is between about 25 mm and 100 mm.

32. The catheter device of claim 28, further comprising:
a seal housing disposed on an outer surface of the return lumen and enclosing a portion of the return lumen and fluid supply lumen at the point at which the fluid supply lumen exits the return lumen.

33. The catheter device of claim 32, wherein the seal housing is disposed on an exterior radius of curvature of the return lumen.

34. The catheter device of claim 32, further comprising:
a seal element disposed within a cavity of the seal housing and around the fluid supply lumen.

35. The catheter device of claim 34, wherein the seal element comprises an o-ring.

36. A uterine lavage catheter device for recovering blastocysts from a human uterus, comprising:
a seal element configured to provide a sealing surface against the external cervical ostium;
a supply lumen extending from the seal element and configured to supply lavage fluid through a nozzle;
a return lumen having an inlet; and
an extension element extending from and in contact with the seal element, the extension element further comprising a distal surface that comprises the sealing surface, the extension element configured to shorten the distance between the inlet of the return lumen and the internal cervical ostium.

37. The catheter device of claim 36, wherein the distal surface of the extension element is conical.

38. A uterine lavage catheter device for recovering blastocysts from a human uterus, comprising:
a seal element configured to provide a sealing surface against the external cervical ostium;
a supply lumen extending from the seal element and configured to supply lavage fluid;
a nozzle configured to generate a spray pattern, wherein the nozzle comprises a first fluid outlet; and a second fluid outlet angled from the first fluid outlet with respect to the axis of the fluid supply lumen; and
a return lumen.

39. The catheter device of claim 38, wherein the angle between the first fluid outlet and the second fluid outlet is between about 0 degrees and 60 degrees

40. A method of recovering blastocysts from a human uterus, comprising:
inserting a catheter device trans-vaginally into a uterus;
sealing the exterior cervical ostium with a sealing surface of a seal element;
lavaging the uterine walls with lavage fluid with a nozzle at a first fluid pressure, wherein the lavaging comprises pulsing lavage fluid between the first fluid pressure and a second fluid pressure different than the first fluid pressure;
placing an inlet of a return lumen at the interior cervical ostium; and
recovering the lavage fluid and blastocysts from the uterus with the return lumen, wherein the first fluid pressure is between about 20 PSI and 50 PSI.

41. A uterine lavage catheter device for recovering blastocysts from a human uterus, comprising:
a seal element configured to provide a sealing surface against the external cervical ostium;
a supply lumen extending from the seal element and configured to supply lavage fluid;
a nozzle configured to generate a spray pattern; and
a return lumen; and
a tip direction fitting, configured to be coupled to one of the supply lumen or return lumen, the tip direction fitting providing a pre-bend to the catheter such that the angle of the catheter tip is fixed and offset from the longitudinal axis of the catheter.

42. A uterine lavage catheter device for recovering blastocysts from a human uterus, comprising:
a seal element configured to provide a sealing surface against the exterior cervical ostium;
a supply lumen extending from the seal element and configured to supply lavage fluid;
a nozzle configured to generate a spray pattern;
a return lumen; and
an imaging sensor configured to image a portion of the uterus.

43. The catheter device of claim 42, wherein the imaging sensor comprises a CCD sensor.

44. The catheter device of claim 42, wherein the imaging sensor is integrated into the nozzle.

45. The catheter device of claim 42, further comprising:
a light source, the light source configured to illuminate a portion of the uterus configured to be imaged by the imaging sensor.

46. A process for recovering blastocysts from a human uterus, comprising:
inserting a catheter device trans-vaginally into a uterus, wherein the catheter device comprises an imaging sensor configured to image a portion of the uterus;
sealing the exterior cervical ostium with a sealing surface of a seal element;
imaging a portion of the uterus with the imaging sensor;
lavaging the uterine walls with lavage fluid with a nozzle;
placing an inlet of a return lumen at the interior cervical ostium; and
recovering the lavage fluid and blastocysts from the uterus via the return lumen.

47. The process of claim 46, further comprising:
illuminating a portion of the uterus to be imaged with a light source coupled to the catheter device.

48. An uterine lavage catheter device for recovering blastocysts from a human uterus, comprising:
a seal element comprising a sealing surface configured to be disposed against an exterior cervical ostium;
a supply lumen extending from the seal element and configured to supply lavage fluid;
a nozzle coupled to the supply lumen;
a return lumen having an inlet, the return lumen disposed coaxially with the supply lumen; and
an extension assembly operatively connected to the seal element and configured to move the seal element in a longitudinal direction along the uterine lavage catheter device to shorten a distance between the inlet of the return lumen and an interior cervical ostium of the human uterus.

49. The uterine lavage catheter device of claim 48, wherein the extension assembly comprises:
a first tube disposed coaxially with the supply lumen and the return lumen and housing a portion of the supply lumen and the return lumen; and
a second tube disposed coaxially with the first tube and connected to seal element, the second tube being configured to move along the first tube such that the second tube moves the seal element in the longitudinal direction along the uterine lavage device.

50. The uterine lavage catheter device of claim 49, wherein the extension assembly comprises:
an actuator disposed coaxially with the first tube and connected to the second tube, the actuator is configured to translate rotary movement about the first tube to linear movement of the second tube to trigger movement of the seal element.

51. The uterine lavage catheter device of claim 50, wherein the actuator comprises a locking pin, and the first tube comprises a plurality of locking notches disposed in the longitudinal direction along an outer surface of the first tube,
wherein each of the locking notches is configured to receive the locking pin as the actuator rotates about the first tube, such that the plurality of notches limits movement of the actuator in incremental positions.

52. The uterine lavage device of claim 51, wherein the extension assembly further comprises:
a helical-shaped spring disposed between the first tube and the actuator, the spring biased in axial direction such that the spring is configured to engage the actuator and limit movement of the actuator.

53. The uterine lavage device of claim 48, wherein the sealing surface comprises a conical-shaped surface disposed at a distal end of the seal element.

54. The uterine lavage device of claim 53, wherein the extension assembly comprises a flexible tube connected to a proximal end of the seal element and configured to move in a longitudinal direction along the uterine lavage device to move the seal element.

55. An uterine lavage catheter device for recovering blastocysts from a human uterus, comprising:
a seal element comprising a vacuum port and a sealing surface configured to be disposed against a cervix surface surrounding an exterior cervical ostium of the human uterus;
a supply lumen extending from the seal element and configured to supply lavage fluid;
a nozzle coupled to the supply lumen;
a return lumen having an inlet, the return lumen disposed coaxially with the supply lumen, and
a vacuum lumen connected to the vacuum port and in communication with a vacuum source,
wherein the vacuum lumen is configured to exhaust air from the seal element such that the sealing surface is configured to be pressure fitted against the cervix surface surrounding the exterior cervical ostium of the human uterus.

56. The uterine lavage catheter device of claim 55, wherein the seal element comprises a cup disposed at a distal end of the seal element, the cup defining the sealing surface.

57. The uterine lavage catheter device of claim 56, wherein the cup comprises a rim and a hemispherical-shaped wall terminating at the rim and defining a cavity, and the vacuum port is disposed on the hemispherical-shaped wall and configured to exhaust air from the cavity via the vacuum lumen.

58. The uterine lavage catheter device of claim 55, wherein the seal element is comprised of a low durometer elastomeric material.

59. The uterine lavage catheter device of claim 58, wherein the seal element is moveable in a longitudinal direction along the uterine lavage catheter device.

60. The uterine lavage catheter device of claim 59, further comprising:
an extension assembly operatively connected to the seal element and configured to move the seal element in a longitudinal direction along the uterine lavage catheter device to shorten a distance between the inlet of the return lumen and an interior cervical ostium of the human uterus.

61. An uterine lavage catheter system for recovering blastocysts from a human uterus, comprising:
a uterine lavage catheter device comprising:
a seal element comprising a sealing surface configured to be disposed against an exterior cervical ostium,
a supply lumen extending from the seal element and configured to supply lavage fluid,
a nozzle coupled to the supply lumen, and
a return lumen having an inlet,
a collection container disposed external to the catheter device and defines a fluid head to control intrauterine pressure and uterine expansion of the human uterus,
an elevator comprising:
a track, and
a cradle coupled to the track and configured to receive and hold the collection container,
wherein the elevator is configured to raise or lower the cradle holding the collection container along the track to adjust intrauterine pressure of the human uterus.

62. The uterine lavage catheter system of claim 61, wherein the elevator comprises a toothed belt drive assembly operatively connected to the track and the cradle and configured to raise and lower the cradle along the track.

63. The uterine lavage catheter system of claim 61, wherein the elevator comprises a screw drive assembly operatively connected to the track and the cradle and configured to raise and lower the cradle along the track.

64. The uterine lavage catheter system of claim 61, wherein the elevator comprises a rack gear assembly operatively connected to the track and the cradle and configured to raise and lower the cradle along the track.

65. The uterine lavage catheter system of claim 61, wherein the collection container is magnetically coupled to the cradle.

66. The uterine lavage catheter system of claim 61, wherein the collection container is coupled to the cradle via direct pin engagement.
